(19) United States
(12) Patent Application Publication
(10) Pub. No.: US 2026/0314989 A1
(43) Pub. Date: Oct. 15, 2026

(54) MULTI-CHANNEL MINIATURE ELECTROENCEPHALOGRAM ACQUISITION SYSTEM AND METHOD, AND ELECTRONIC DEVICE

(71) Applicant: TIANJIN UNIVERSITY, Tianjin (CN)

(72) Inventors: Minpeng XU, Tianjin (CN); Yu CAI, Tianjin (CN); Yongzhi HUANG, Tianjin (CN); Zexu ZHANG, Tianjin (CN); Chunhou ZHENG, Tianjin (CN); Yangyang YE, Tianjin (CN); Hui LI, Tianjin (CN); Dong MING, Tianjin (CN)

(21) Appl. No.: 19/109,307

(22) PCT Filed: Feb. 7, 2024
(86) PCT No.: PCT/CN2024/076812

§ 371 Date: Mar. 6, 2025

(30) Foreign Application Priority Data
Dec. 19, 2022 (CN) 202211632898.5
Dec. 19, 2022 (CN) 202311185548.3
Feb. 3, 2023 (CN) 202310053158.4

(51) Int. Cl. A61B 5/384; A61B 5/00; A61B 5/254; A61B 5/291; A61B 5/31; A61B 5/378

(52) U.S. Cl. CPC A61B 5/384; A61B 5/0024; A61B 5/254; A61B 5/291; A61B 5/31; A61B 5/378; A61B 5/6814; A61B 5/6838; A61B 5/742; A61B 2560/0209; A61B 2560/0214; A61B 2562/046; A61B 2562/166

(57) ABSTRACT

A multi-channel miniature electroencephalogram acquisition system includes multiple single-channel electroencephalogram acquisition devices, a data label synchronization device and a terminal. A stimulation module transmits stimulation label data to the data label synchronization device through a USB interface when a stimulation signal is generated; the data label synchronization device issues clock synchronization information and a control command, and receives the stimulation label data; the multiple single-channel electroencephalogram acquisition devices receive the clock synchronization information, collect EEG data based on the control command, and transmit the EEG data to the data label synchronization device through BLE; the data label synchronization device synchronizes the EEG data and the stimulation label data, and transmits synchronized data and the stimulation label data to the waveform recording module; the waveform recording module displays a waveform of the EEG data corresponding to the stimulation label data and perform data store.

### CROSS-REFERENCE TO RELATED APPLICATIONS

This application is a U.S. national phase entry under 35 U.S.C. § 371 of International Application No. PCT/CN2024/076812, filed on Feb. 7, 2024, which claims priority to Chinese Patent Application No. 202211632898.5, filed on Dec. 19, 2022, Chinese Patent Application No. 202311185548.3, filed on Dec. 19, 2022, and Chinese Patent Application No. 202310053158.4, filed on Feb. 3, 2023. The entire disclosures of the above-identified applications are hereby incorporated herein by reference.

### TECHNICAL FIELD

The application relates to the technical field of electroencephalogram (EEG) acquisition devices, particularly a multi-channel miniature EEG acquisition device that is wearable, miniature, and user-friendly.

### BACKGROUND

Conventional desktop electroencephalogram (EEG) acquisition devices are widely used in laboratories. The conventional devices generally use wet electrodes as acquisition electrodes, so it is necessary to inject conductive gel between the electrodes and the scalp before the experiment, to maintain good contact between them and reduce the interface contact impedance.

### SUMMARY

The disclosure aims to solve one of the technical problems existing in the related art to a certain extent.

The disclosure aims to provide a multi-channel miniature electroencephalogram (EEG) acquisition system, a multi-channel miniature electroencephalogram (EEG) acquisition method, and an electronic device. The disclosure designs a single-channel miniature EEG acquisition circuit. The circuit includes a Bluetooth low energy (BLE) micro controller unit (MCU) for logic control and data transmission, a low power programmable gain analog acquisition front end for EEG signal acquisition, a charge pump with positive and negative voltage outputs for providing power supply to various chips, and a miniature cylindrical battery for providing power supply of the system. The disclosure designs a new common mode signal feedback channel to reduce the use of lead wires. The device has the characteristics of low power consumption, miniaturization, and portability. The disclosure designs a combining a hairpin structure wearing method and an auxiliary electrode fixing method, so that the miniature EEG acquisition device may be fixed on the scalp through the hairpin structure design of the device, which is used together with viscous gel electrodes to enable the electrodes and the scalp to become viscous. The use of the two fixing methods, namely the hairpin and the gel electrodes, has the characteristics of miniaturization, stealthiness, and strong wearability, which are more easily accepted by the consumers. The single-channel miniature EEG acquisition device is designed with a replaceable direct-connected electrode. The replaceable electrode is directly connected to the acquisition circuit by using a printed circuit board (PCB) substrate pad, which reduces the possibility of common mode interference or other noise introduced by long lead wires and reduces the cost of the experiment. The form of the electrode may be a wet electrode, a dry electrode, or a gel electrode. The type of the electrode body may be a disk electrode, an elastic copper ball electrode, or a sintered silver-silver chloride electrode. The disclosure designs a data label synchronization device based on dual serial ports. One of the dual serial ports is configured to receive stimulation label information sent by a stimulation program and the other one of the dual serial ports is configured to send synchronized data label to an upper computer. The data label synchronization device based on dual serial ports reduces the complexity of the acquisition device and improves the accuracy of the labeling process. The disclosure utilizes a communication method of the BLE with one master and a plurality of slaves, and designs a multi-channel EEG acquisition system based on the single-channel miniature EEG acquisition device. A plurality of single-channel miniature EEG acquisition devices collect EEG data, in which the positions of the devices may be set based on the 10-20 standard leads, or the number and the positions of the devices may be set according to the experimental needs, and the connection method may be designed according to the experimental needs, which provides higher scalability and reconfigurability.

According to the first aspect of the disclosure, a multi-channel miniature EEG acquisition system is provided. The system includes: a plurality of single-channel EEG acquisition devices, a data label synchronization device, and a terminal device, in which the terminal device includes a waveform recording module and a stimulation module; the stimulation module is configured to transmit stimulation label data to the data label synchronization device through a universal serial bus (USB) interface in a case that a stimulation signal is generated; the data label synchronization device is configured to issue clock synchronization information and control commands, and receive the stimulation label data; the plurality of single-channel EEG acquisition devices are configured to receive the clock synchronization information, collect EEG data based on the control command, and transmit the EEG data to the data label synchronization device through Bluetooth low energy (BLE); the data label synchronization device is further configured to synchronize the EEG data and the stimulation label data, and transmit synchronized data and the stimulation label data to the waveform recording module; and the waveform recording module is configured to display a waveform of the EEG data corresponding to the stimulation label data and perform data store.

According to the second aspect of the disclosure, a method for data processing is provided. The method includes: obtaining stimulation label data in a case that a stimulation signal is generated; obtaining clock synchronization information, performing clock synchronization, and obtaining a signal acquisition command; collecting EEG data based on the signal acquisition command after the clock synchronization, and synchronizing the EEG data and the stimulation label data; and obtaining a waveform of the EEG data corresponding to the stimulation label data based on the synchronized data and storing the data.

According to the third aspect of the disclosure, an electronic device is provided. The electronic device includes a processor, and a memory storing an embedded program executable by the processor, in which when the embedded program is executed by the processor, the method according to any of the second aspect is implemented.

Additional aspects and advantages of the disclosure will be set forth in part in the description which follows, and in part will be obvious from the description, or will be learned by practice of the disclosure.

### BRIEF DESCRIPTION OF THE DRAWINGS

The above-mentioned and/or additional aspects and advantages of the disclosure will become apparent and readily appreciated from the following description of embodiments in combination with the accompanying drawings.

FIG. 1 is a block diagram illustrating a structure of a multi-channel miniature electroencephalogram (EEG) acquisition system according to an embodiment of the disclosure.

FIG. 2 is a schematic diagram illustrating a steady-state visual evoked potential (SSVEP) single-channel EEG experiment based on dual monitors according to an embodiment of the disclosure.

FIG. 3 is a schematic diagram illustrating a structure of a single-channel miniature EEG acquisition system according to an embodiment of the disclosure.

FIG. 4 is a schematic diagram illustrating a structure of a multi-channel miniature EEG acquisition system according to an embodiment of the disclosure.

FIG. 5 is a block diagram illustrating a structure of an internal circuit system of a single-channel miniature EEG acquisition device according to an embodiment of the disclosure.

FIG. 6 is a schematic diagram illustrating a model of an acquisition circuit board in a single-channel miniature EEG acquisition device according to an embodiment of the disclosure.

FIG. 7 is a schematic diagram illustrating a model of a power supply board in a single-channel miniature EEG acquisition device according to an embodiment of the disclosure.

FIG. 8 is a sectional view of a hairpin structure according to an embodiment of the disclosure.

FIG. 9 is a schematic diagram illustrating a hairpin wearing structure according to an embodiment of the disclosure.

FIG. 10 is a schematic diagram illustrating a structure of a housing of a single-channel miniature EEG acquisition device according to an embodiment of the disclosure.

FIG. 11 is a schematic diagram illustrating another structure of a housing of a single-channel miniature EEG acquisition device according to an embodiment of the disclosure.

FIG. 12 is a schematic diagram illustrating a hair fixing structure of a single-channel miniature EEG acquisition device according to an embodiment of the disclosure.

FIG. 13 is a schematic diagram illustrating a structure of a hairpin according to an embodiment of the disclosure.

FIG. 14 is a schematic diagram illustrating a flip baseline structure according to an embodiment of the disclosure.

FIG. 15 is a schematic diagram illustrating electrodes of different materials according to an embodiment of the disclosure.

FIG. 16 is a schematic diagram illustrating a replaceable electrode according to an embodiment of the disclosure.

FIG. 17 is a schematic diagram illustrating a bottom of an electrode substrate according to an embodiment of the disclosure.

FIG. 18 is a schematic diagram illustrating a structure of an electrode with a signal input according to an embodiment of the disclosure.

FIG. 19 is a schematic diagram illustrating a Laplace dry electrode with 2 input signal ends according to an embodiment of the disclosure.

FIG. 20 is a block diagram illustrating a structure of an internal circuit system of a data label synchronization device according to an embodiment of the disclosure.

FIG. 21 is a schematic diagram illustrating command transmission according to an embodiment of the disclosure.

FIG. 22 is a schematic diagram illustrating an SSVEP single-channel EEG experiment based on dual monitors according to an embodiment of the disclosure.

FIG. 23 is a schematic diagram illustrating data acquisition of a multi-channel miniature EEG acquisition device according to an embodiment of the disclosure.

FIG. 24 is a schematic diagram illustrating connections of a multi-channel miniature EEG acquisition device according to an embodiment of the disclosure.

FIG. 25 is a flow chart illustrating an implementation of an EEG acquisition process according to an embodiment of the disclosure.

FIG. 26 is a flow chart illustrating an implementation of a process of EEG acquisition according to an embodiment of the disclosure.

FIG. 27 is a schematic diagram illustrating a common reference connection method of multi-channel miniature EEG acquisition devices according to an embodiment of the disclosure.

FIG. 28 is a flow chart illustrating a method for data processing according to an embodiment of the disclosure.

FIG. 29 is a schematic diagram illustrating a structure of a housing according to an embodiment of the disclosure.

FIG. 30 is a schematic diagram illustrating a structure of a hairpin wearing structure according to an embodiment of the disclosure.

FIG. 31 is a flow chart illustrating a data synchronization process of a method for data processing according to an embodiment of the disclosure.

FIG. 32 is a schematic diagram illustrating a time relationship of a data synchronization process of a method for data processing according to an embodiment of the disclosure.

FIG. 33 is a flow chart illustrating a data synchronization process of a method for data processing according to an embodiment of the disclosure.

FIG. 34 is a block diagram illustrating a structure of an electronic device according to an embodiment of the disclosure.

### DETAILED DESCRIPTION

It should be noted that embodiments and features of embodiments of the disclosure may be combined with each other without conflict. The disclosure will be explained in detail below in combination with embodiments with reference to the accompanying drawings.

In order to enable those skilled in the art to better understand the embodiments of the disclosure, the technical solutions in the embodiments of the disclosure will be described clearly and completely in the following, in combination with the accompanying drawings in the embodiments of the disclosure. Obviously, the described embodiments are only a part of the embodiments of the disclosure, and not all of the embodiments. Based on the embodiments in the disclosure, all other embodiments obtained by a person of ordinary skill in the art without making creative labor shall fall within the scope of protection of the disclosure.

Conventional desktop electroencephalogram (EEG) acquisition devices can only be widely used in well-controlled laboratories for following limitations. Firstly, the conventional devices are large in size and heavy in weight, and require Alternating Current (AC) power supply, which may cause power-line interference, and limits their outdoor applications. Secondly, the conventional devices generally use wet electrodes as acquisition electrodes, so it is necessary to inject conductive gel between the electrodes and the scalp before the experiment, to maintain good contact between them and reduce the interface contact impedance. This may increase the preparation of the experiment. At the same time, after the experiment, the hair and the EEG cap need to be cleaned. The experimental process is relatively cumbersome. Finally, the use of long wires to connect the EEG cap and the amplifier may increase the possibility of introducing common mode interference or other noise, increasing the complexity of subsequent data processing.

Although the methods in the related art may confront technical problems such as the following in collecting EEG signals using conventional EEG acquisition devices.
1. The conventional desktop EEG acquisition device cannot be put into practical application since the device, which often uses EEG cap-type acquisition electrodes, has a large size and powered by AC power supply, and may only be applied in the laboratory. The portable EEG devices, which are still relatively large in volume and weight, use headbands, helmets and other fixation methods that make the devices less stealthy and wearable. Thus, the portable EEG devices are not easily accepted by the consumers.
2. The EEG acquisition devices use long wires to connect the electrodes to the amplifier, which increases the possibility of introducing common mode interference or other noise, and increases the complexity of subsequent data processing.
3. The wireless portable EEG devices require a matching router and a labeling box device when performing experiments with stimulation labels such as a steady-state visual evoked potential (SSVEP) experiment, which increases the complexity of the acquisition devices.
4. The position of the acquisition electrodes of the EEG caps, the headbands, and the helmets is fixed and cannot be changed, which makes the experiment inflexible and poor in scalability and reconfigurability.

A multi-channel miniature electroencephalogram (EEG) acquisition system and method according to embodiments of the disclosure are described below with reference to the accompanying drawings.

FIG. 1 is a block diagram illustrating a structure of a multi-channel miniature EEG acquisition system according to an embodiment of the disclosure.

As shown in FIG. 1, the system 100 includes: a plurality of single-channel EEG acquisition devices 1, a data label synchronization device 2, and a terminal device 3. The terminal device includes a waveform recording module and a stimulation module.

The stimulation module is configured to transmit stimulation label data to the data label synchronization device 2 through a universal serial bus (USB) interface in a case that a stimulation signal is generated.

The data label synchronization device 2 is configured to issue clock synchronization information and a control command, and receive the stimulation label data.

The plurality of single-channel EEG collection devices 1 are configured to receive the clock synchronization information, collect EEG data based on the control command, and transmit the EEG data to the data label synchronization device 2 through Bluetooth low energy (BLE).

The data label synchronization device 2 is further configured to synchronize the EEG data and the stimulation label data, and transmit synchronized data and the stimulation label data to the waveform recording module.

The waveform recording module is configured to display a waveform of the EEG data corresponding to the stimulation label data and perform data store.

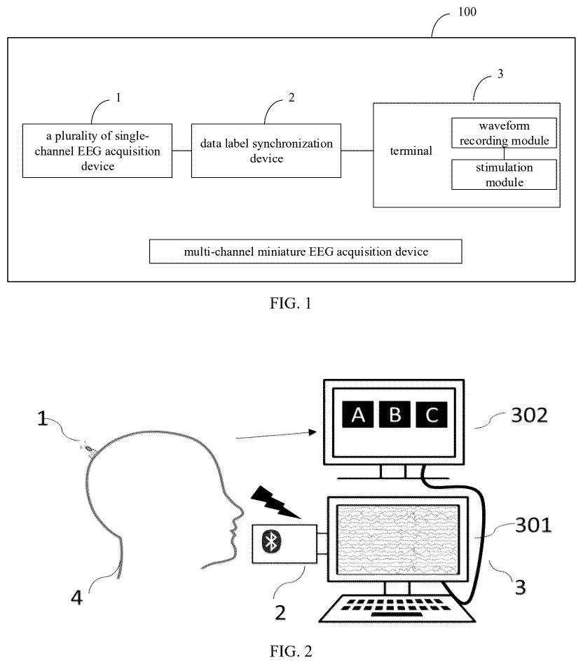

As shown in FIG. 2, a participant (4) (selected by an experimenter) participates in an EEG experiment in which the participant watches a stimulation program (302) on a display screen, which stimulates EEG signals such as the SSVEP. (in which the serial port 1 performs a setup and operation of the acquisition device, and observes an EEG waveform of the participant). The single-channel EEG acquisition device (1) is configured to collect EEG data and send the EEG data to the data label synchronization device (2) through a BLE. A sampling rate may be set to 250 Hz-8 KHz, and the number of the devices may be configured to 32 according to demands. The data label synchronization device (2) is configured to communicate with the single-channel EEG acquisition device (1) through the BLE, and communicate with an upper computer (3) through a USB structure, to receive a control command from a waveform recording program (301) of the upper computer (3) and forward the command to the device (1). The device (2) is further configured to receive EEG data from the device (1) and the stimulation program (302) of the upper computer (3), and forward the data and the program to the waveform recording program (301) of the upper computer (3), after performing the label and data synchronization. The upper computer software (3) includes a waveform recording program (301) and a stimulation program (302). The upper computer is the same as the terminal device mentioned above. The waveform recording program (301) is configured to record the EEG signal waveform, issue the control command, and store the EEG data. The stimulation program (302) is configured to display a stimulation interface, and the stimulation program (302) sends stimulation label information to the device (2) before flashing stimulation is started. The waveform recording program (301) may not be limited to on a personal computer (PC) side. The waveform recording program (301) may be on a cell phone side or a VR side. The stimulation program (302) may not be limited to on the PC side. The stimulation program (302) may be on the cell phone side, the VR side, or may be on an embedded terminal device. For example, the stimulation program (302) may be a light emitting diode (LED) flashing with different frequencies.

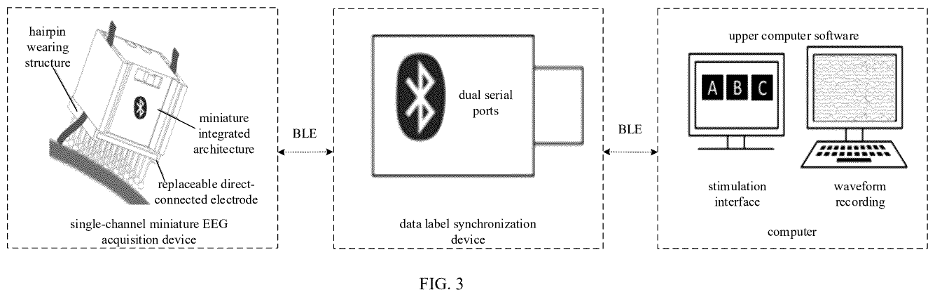

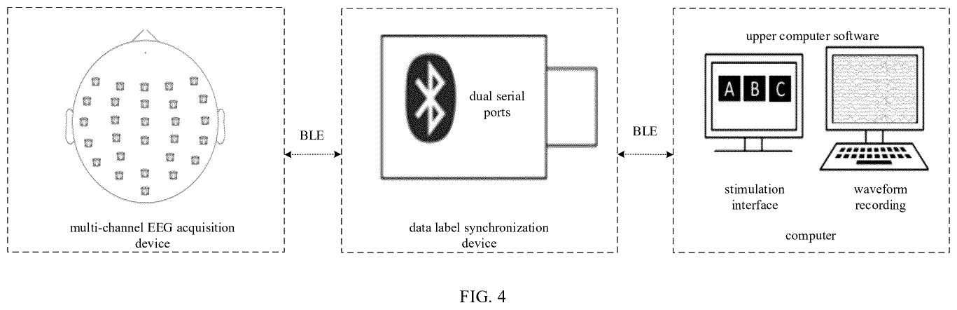

Further, as shown in FIG. 3 and FIG. 4, FIG. 3 is a schematic diagram illustrating a structure of a single-channel miniature EEG acquisition system, and FIG. 4 is a schematic diagram illustrating a structure of a multi-channel miniature EEG acquisition system.

Further, a single-channel miniature EEG acquisition device includes a single-channel miniature EEG acquisition circuit. The circuit includes a BLE micro controller unit (MCU) for logic control and data transmission, a low power programmable gain analog acquisition front end for EEG signal acquisition, a charge pump with positive and negative voltage outputs for providing power supply to various chips, and a miniature cylindrical battery for providing power supply to the system. A new common mode signal feedback channel is designed to reduce the use of lead wires. A design of a miniature modular 4-layer PCB allows the size of the acquisition device to be within 1 cm³, which has characteristics of low power consumption, miniaturization, and portability.

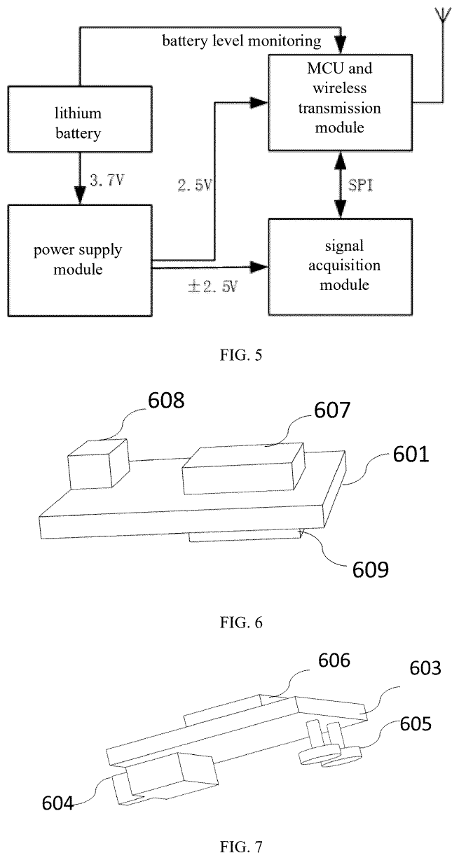

As shown in FIG. 5, the single-channel miniature EEG acquisition circuit includes a Microcontroller Unit (MCU) and wireless transmission module, a signal acquisition module, a power supply module, and a lithium battery.

The MCU and wireless transmission module is a BLE MCU and a circuit thereof, which is configured to read the data collected by the signal acquisition module, and forward the data to the data label synchronization device through the BLE, and monitor a battery level. The MCU and wireless transmission module may be CC2640R2FRSM. The signal acquisition module is an integrated analog front end acquisition chip and a circuit thereof, which is configured to collect EEG signals. The analog front end acquisition chip may be an ADS129x chip of TI, such as ADS1291. The sampling rate may be set to 250 Hz-8 KHz. The signal acquisition module communicates with the MCU and wireless transmission module through a serial peripheral interface (SPI). The signal acquisition circuit adopts a two-wire differential signal design, and the two-wire differential signal includes a non-inverting input end and inverting input end. It is further explained that the two-wire differential signal is extended to two points on the housing of the acquisition device by short wires and conductive tape or copper foil, to directly connect to the pads of the acquisition electrodes. The signal acquisition circuit designs a new common mode signal feedback circuit, which feeds the common mode interference of the differential input signal back to the signal input through two feedback resistors. The new feedback circuit saves one feedback lead wire compared to the conventional EEG acquisition system. The power supply module is a voltage converter and a circuit thereof. The voltage converter converts a lithium battery voltage into a positive voltage and a negative voltage, to supply power to the signal acquisition module and the MCU and wireless transmission module. The converted voltage may be ±2.5 V. The voltage converter may be TI LM27762. The lithium battery is a rechargeable battery that is connected to the power supply module to power the system. The lithium battery may be a cylindrical (button) rechargeable battery.

Further, to minimize the size, the single-channel miniature EEG acquisition circuit system may be designed with 2 PCBs, both less than 10 mm in length and width. As shown in FIG. 6, the signal acquisition module 609 and the MCU and wireless transmission module 607 are designed on two sides of one PCB. The resistance capacitance may be a 0201 package, and the antenna is selected to be a small-sized ceramic antenna 608. The PCB is referred to as the acquisition circuit board 601.

The acquisition circuit board 601 adopts a multi-layer circuit board design to reduce interference/leakage effects and to reduce the transmission loss of the microstrip line. The multi-layer circuit board design may be a four-layer board design with a board layer structure of a radio frequency signal layer, a ground layer, a power supply layer, and a signal layer.

As shown in FIG. 7, the power supply module 606 is designed on one PCB with dimensions of 10 mm*10 mm. The PCB is referred to as the power supply board 603. The power supply board 603 has two PogoPin electrodes 605, configured to charge electrodes, and a toggle switch 604, configured to switch the power supply of the system. The power supply board 603 is connected to the cylindrical rechargeable lithium battery 602 and converts the voltage of the cylindrical rechargeable lithium battery 602 to a voltage of ±2.5V. The power supply board 603 is connected to the acquisition circuit board 601 by 3 thin wires and provides ±2.5V voltage to the acquisition circuit board 601.

In an embodiment, the disclosure further designs steps for fixing the single-channel EEG acquisition device. The steps include: obtaining a fixing method of the single-channel EEG acquisition device; in which the fixing method includes a fixing method combining a hairpin wearing structure and an auxiliary electrode; in which the hairpin wearing structure includes a hardware fixing structure and a hairpin structure; and fixing hairs of a participant using a groove of the hairpin structure, the hardware fixing structure and the auxiliary electrode, to fix the single-channel EEG acquisition device on the scalp of the participant to collect an EEG signal of the participant.

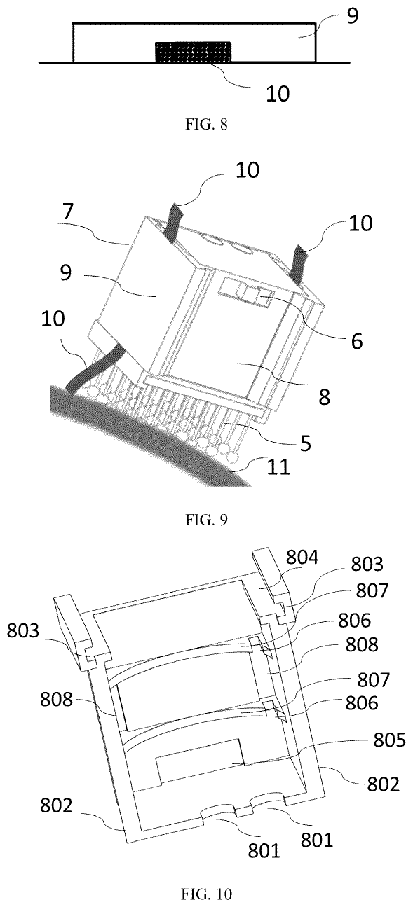

In particular, the wearing method of the EEG acquisition device of the single-channel miniature EEG acquisition device 1 includes wearing a hairpin and fixing by the auxiliary electrodes. The auxiliary electrodes, for example, may be fixed using a gel electrode, or other solid electrodes with viscosity may be used for assisting in fixing. It may be understood that the auxiliary electrodes are configured to increase signal conduction and enhance fixation effects. The auxiliary electrodes may also be used alone for fixing because the electrodes are very light. In the case that the weight of the electrode is less than 5 g, the electrode may be fixed by using a conductive gel alone. Conductive gel such as TEN20 conductive gel may be used alone for fixing. The device may also be fixed by using the hair fixing method and the electrodes without viscosity. The hairpin wearing structure includes a hardware fixing structure 8 and a hairpin structure 9. The hardware fixing structure is connected to the acquisition electrodes, fixes the internal circuit board and the battery. The hairpin structure 9 fixes the miniature EEG acquisition device to the scalp by a groove designed to contain a sponge tape to lock a certain amount of hair 10. The hairpin structure 9 is used in combination with gel electrodes that are viscous, so that the electrodes and the scalp become viscous. This method has the characteristics of miniaturization, stealthiness, and wearability, thus it is more easily accepted by the consumers. The hairpin structure may be as shown in FIG. 8.

Further, an embodiment of the hairpin wearing structure is shown in FIG. 9.

The hairpin wearing structure 9 includes a hardware fixing structure 8 that is configured to connect the acquisition electrodes 5, fix the internal circuit boards (601, 603) and the battery 602, and a hairpin structure 9 that is configured to fix the acquisition device to the scalp 11 by fixing the hair 10.

As shown in FIG. 10, the hardware fixing structure refers to a rational layout and fixing of the acquisition electrodes, the internal circuit, and the battery by designing the housing 8. The hardware fixing structure includes an acquisition electrode fixing structure, an internal circuit structure and a battery fixing structure.

The housing 8 has an electrode substrate fixing and connecting structure. The disclosure designs a groove structure 803 that is configured to fix the electrode 5 with a square substrate.

The groove of the groove structure includes conductive tape or copper foil 804 connected with a signal line of an acquisition circuit board 601. The electrode substrate 501 is pushed into the groove 803 to fix the electrode body 5. The copper foil 804 is connected to the exposed PCB pads at the bottom of the electrode to realize the conduction between the acquisition electrode and the acquisition circuit.

The housing 8 is designed with a three-internal-layer structure that are configured to fix the internal circuit boards (601, 603) and the battery 602.

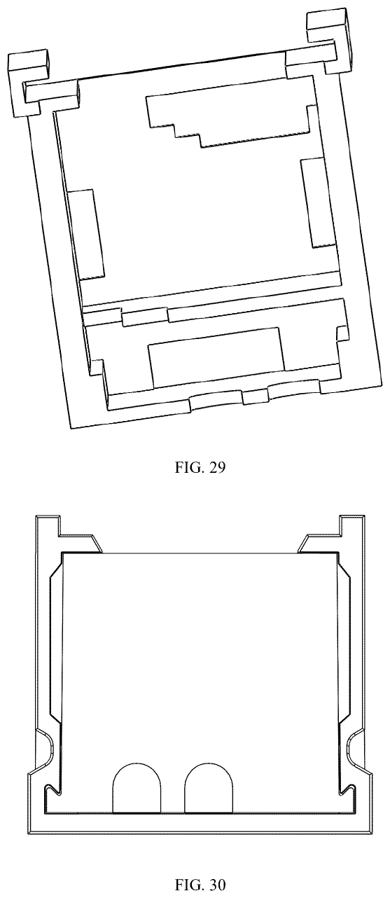

As shown in FIG. 29, the three internal layers structure of the housing 8 is a one-piece molding design with a hollow circular design in the middle which forms a protruding card structure 807. The card structure 807 divides the inside of the housing into three layers, in which the power supply board 603, the battery 602, and the acquisition circuit board 601 are placed in the three layers of housing 8 respectively. The power supply board 603, the battery 602, and the acquisition circuit board 601 are fixed and separated. The card structure 807 is provided with a rectangular empty groove 806 configured to pass a connection line between the acquisition circuit board 601 and the power supply 603.

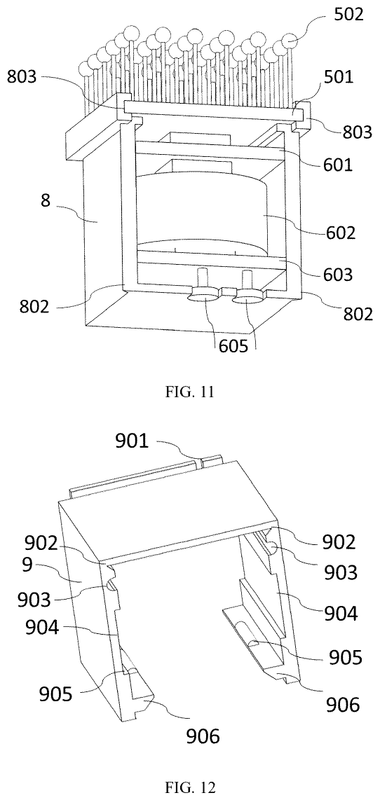

As shown in FIG. 11, a lower aperture 805 of the housing 8 is a switch interface. A semicircular hole 801 is provided in the bottom of the housing 8, configured to settle the two PogoPin electrodes 605 of the power supply board 603.

Preferably, as shown in FIG. 30, another hairpin wearing structure is designed. One difference from the above structure is that the hairpin wearing structure is a bent structure, which is transformed from type circular into a body-like type. Another difference is that the structure for fixing the slipcover and the housing is an inverted triangle structure, which increases the stability of the structure.

The back of the housing 8 is provided with 2 rectangular holes 808 that are configured to lock the semi-cylindrical structure 905 of the hairpin structure 9. The triangular elongated structures 802 on both sides of the open side of the housing 8 are configured to fill the triangular grooves 902 of the hairpin structure 9, thus closing the housing 8 and fixing the hairpin structure 9.

As shown in FIG. 12, the hairpin structure 9 has a hair fixing structure. For example, according to the present disclosure, the miniature EEG acquisition device may be fixed to the scalp by designing a groove containing a sponge tape to lock a certain amount of hair or by increasing the friction between the hair and the device in other ways.

The hairpin structure 9 is a one-piece molding design. The hairpin structure 9 has triangular grooves 902 on both sides of the top, which are connected to the fixing connection structures 802 on both sides of the open side of the housing 8 by sliding. The fixing connection structure may be a triangular elongated structure, a square elongated strip, a semi-circular structure, or any connection structure of a plurality of shapes that may closely connect the housing to the hair fixing structure.

As shown in FIG. 14, the hairpin structure 9 has a flip baseline point which may be opened to both sides. The disclosure adopts a semi-circular groove 903, and may also adopt a circular and cylindrical rotation pivot structure as a flip baseline point.

The hairpin structure 9 has a hair fixing structure that is configured to lock the hair 10. The disclosure adopts a groove 904 in which a material with a high friction is provided to increase the friction between the hair and structure for a more reliable fixing. Preferably, the material may be a sponge tape.

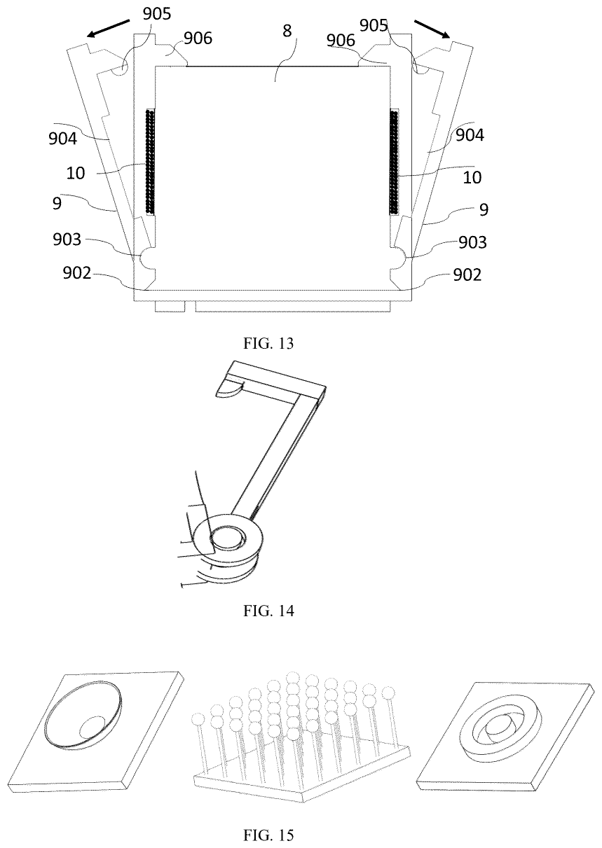

As shown in FIG. 13, the bottom of the groove 904 is a buckle 906, configured to lock the housing 8. The upper of the buckle is provided with a semi-cylindrical structure 905, configured to lock the 2 rectangular holes 808 at the back of the housing 8, thus fixing the housing 8 together with the hair 10 and the hairpin structure 9.

The specific steps of wearing may include: opening the buckle 906 of the hairpin structure 9 to the two outer sides, in which the buckle 906 may open to the outer side with the semi-circular groove as a pivot point; putting a certain amount of hair 10 into the groove 904, adjusting the height of the acquisition device 1 by applying downward pressure for a good connection between the electrodes and the scalp; and closing the buckle 906 inwardly to fix the acquisition device 1 to the scalp.

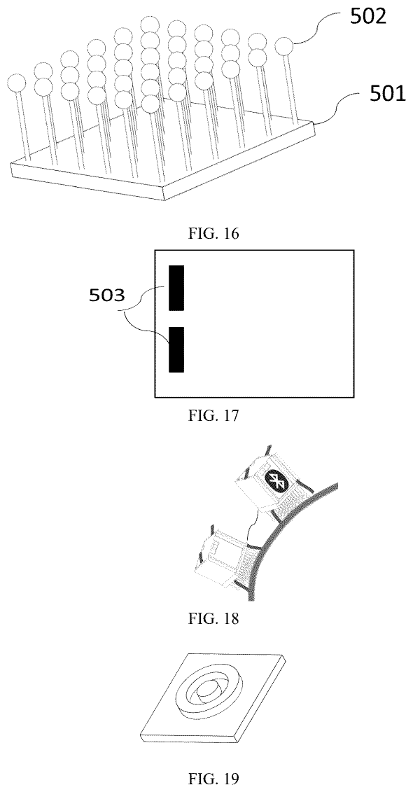

Further, the single-channel miniature EEG acquisition device is designed with a replaceable direct-connected electrode, which reduces the possibility of introducing common mode interference or other noise introduced by long lead wires. The replaceable electrode reduces the cost of the experiment. The form of the electrode may be a wet electrode, a dry electrode, or a gel electrode. The replaceable electrode includes an electrode body and an electrode substrate, in which the electrode body includes one of a disk electrode, an elastic copper ball electrode, or a sintered silver-silver chloride electrode. As shown in FIG. 15, the replaceable direct-connected electrode has a square electrode substrate which is designed with a PCB. The electrode is replaceable as the electrode substrate 501 is pushed into the groove structure of the housing 8 to be fixed. The replaceable direct-connected electrode realize the conduction between the acquisition electrode and the acquisition circuit and the direct connection between the electrode and the acquisition circuit by pushing the electrode substrate into the groove structure of the housing 8 to be fixed, to connect the exposed PCB pads of the substrate to the copper foil 804 in the groove structure 803 that is connected to the signal line of the acquisition circuit board 601. As shown in FIG. 16, the replaceable direct-connected electrode has 1 or 2 signal inputs to obtain 1 or 2 signals. The replaceable direct-connected electrode has a square base. As shown in FIG. 17, the square base is designed with a PCB. And the bottom of the square base is provided with one or two exposed PCB pads 503 according to the number of signal inputs and is connected to the signal inputs. The electrode with one signal input requires two electrodes for the acquisition of the EEG signals. The two electrodes are connected to the non-inverting input and the inverting input respectively of the two differential input signals of the signal acquisition circuit. One electrode is connected to the non-inverting input of the signal acquisition circuit through the PCB pad 503. And the other electrode is connected to the inverting input of the signal acquisition circuit through a short wire which does not have an acquisition circuit. The two electrodes are configured to realize the acquisition of the EEG signal. As shown in FIG. 18, the electrode with two signal inputs requires only one electrode for the acquisition of the EEG signals. The two inputs of the electrode are connected to the non-inverting input and the inverting input of the acquisition circuit through the PCB pad 503 to realize the acquisition of the EEG signals. The disclosure prefers the Laplace dry electrode with 2 signal inputs.

As shown in FIG. 19, the material of the Laplace dry electrode with two signal inputs may be preferred as sintered silver-silver chloride. The ring and the center of the circle are sintered silver-silver chloride having a certain height in the range of 1 mm-10 mm, and preferably, the height may be 3 mm.

Further, the data label synchronization device is designed with dual serial ports that are configured to synchronize and forward the data and stimulation labels respectively. The data label synchronization device, acting as a master computer, communicates with the single-channel EEG acquisition device through the BLE, and communicates with the waveform recording program and the stimulation program of the upper computer through the USB interface. The data label synchronization device forwards the data and command to the single-channel miniature EEG acquisition device and the upper computer, and synchronizes the EEG data and stimulation label information, which is then forwarded to the waveform recording program of the upper computer.

In particular, the device 2 for data label synchronization is designed with dual serial ports. The serial port 1 is configured to receive a command sent by the waveform recording program 301 of the upper computer 3 and to forward the command to the single-channel miniature EEG acquisition device. The serial port 2 is configured to receive the stimulation label sent by the stimulation program 302 of the upper computer 3; receive the EEG data of the single-channel EEG acquisition device 1 and synchronize the EEG data with the stimulation label, and forward the EEG data to the waveform recording program of the upper computer 3 through the serial port 1. The stimulation label may be further described as, e.g., the label information of the different flashing boxes that start flashing when the SSVEP test is performed, and 1, 2, 3 . . . may be configured to represent the label information of which stimulus box starts flashing.

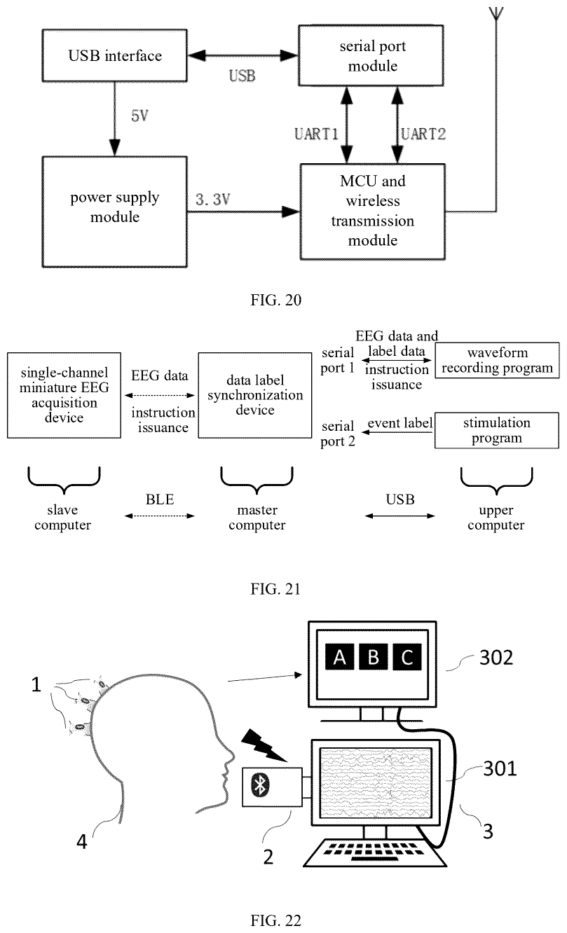

As shown in FIG. 20, the device 2 for data label synchronization includes a MCU and wireless transmission module, a serial port module, and a power supply module.

The MCU and wireless transmission module of the device 2 is a BLE MCU, which communicates with the plurality of single-channel miniature EEG acquisition devices 1 through the BLE. The MCU and wireless transmission module is configured to receive and synchronize the data of the plurality of devices 1. The MCU and wireless transmission module may be a CC26xx BLEMCU, and is preferred as CC2642R. Preferably, the preferred CC2642R chip may realize up to 32-lead lead measurements.

The serial port module of the device 2 connects the MCU and wireless transmission module with the PC upper computer 3. The serial port module is configured to transmit the EEG data synchronized by the MCU and wireless transmission module, the control command of the upper computer and the stimulation label.

The serial port module is a dual serial ports circuit. The serial port 1 communicates with the waveform recording program 301 of the upper computer 3, in which the serial port 1 is configured to transmit the data of the acquisition signal and the control command for the acquisition signal. The serial port 2 communicates with the stimulation program 302 of the upper computer 3, in which the second serial port circuit is configured to receive the stimulation label data. The dual serial ports circuit may be a multi-serial ports chip, which may be CH9344, with a baud rate of up to 12 Mbps to reduce the stimulation labeling delay.

The power supply module uses the USB interface of the PC to provide 5V voltage and the power supply module provides 3.3V power for the main control and wireless transmission module and the serial port module. Preferably, the TPS61025DRCR converts 5V voltage to 3.3V voltage.

The device 2 is designed with a multi-layer circuit board to reduce interference/leakage effects and to reduce the transmission loss of the microstrip line. Preferably, the multi-layer circuit board may be a four-layer board design with a board layer structure of a radio frequency signal layer, a ground layer, a power supply layer, and a signal layer.

Further, as shown in FIG. 21, the upper computer software includes a waveform recording program and a stimulation program. The waveform recording program is configured to record the EEG signal waveform, the issuance of the control command, and the storage of the EEG data. The stimulation program is configured to displaying a stimulation interface, and the stimulation program sends stimulation label information to the data label synchronization device before flashing stimulation is started. The waveform recording program may not be limited to on a PC side. The waveform recording program may be on a cell phone side or a VR side. The stimulation program may not be limited to on the PC side. The stimulation program may be on the cell phone side, the VR side, or may be on the embedded side of the device, e.g., the stimulation program is configured for different frequencies of LED flashing.

The upper computer 3 program includes a waveform recording program 301 and a stimulation program 302, which may be displayed separately by 2 monitors. The upper computer 3 program communicates with the data label synchronization device through the USB interface. The waveform recording program 301 accepts the EEG data and the label information transmitted by the data label synchronization device through the serial port 1, and performs waveform display and data storage. The user issues commands to the single-channel miniature EEG acquisition device through the waveform recording program 301. The stimulation program 302 is configured to display a stimulation interface on which the participant is gazing, and the stimulation program 302 sends stimulation label start information to the device 2 for data label synchronization through the serial port 2 before flashing stimulation is started.

Further, the multi-channel miniature EEG acquisition system utilizes a communication method of BLE with one master and multiple slaves. A plurality of single-channel miniature EEG acquisition devices acquire EEG data, and the position of the devices may be placed according to the 10-20 standard leads. Alternatively, the number and the position of the devices may be placed according to the experimental needs, and the connection method may be designed according to the experimental needs, which provides a higher scalability and reconfigurability.

Preferably, a new and expandable multi-lead EEG acquisition method based on BLE transmission includes a plurality of single-channel miniature EEG acquisition devices 1, a data label synchronization device, and an upper computer 3.

As shown in FIG. 22, a plurality of single-channel miniature EEG acquisition devices serve as a plurality of slave computers, and a data label synchronization device serves as a master computer based on the communication method of BLE with one master and multiple slaves.

The plurality of single-channel miniature EEG acquisition devices 1 communicate with the data label synchronization devices 2 through the BLE. The devices 2 communicates with the upper computer through a USB interface and transmits the data and the command to the plurality of devices 1 and the upper computer 3. The upper computer 3 includes a waveform recording program 301 for recording the EEG data and a stimulation program 302 for generating the stimulation and the stimulation label.

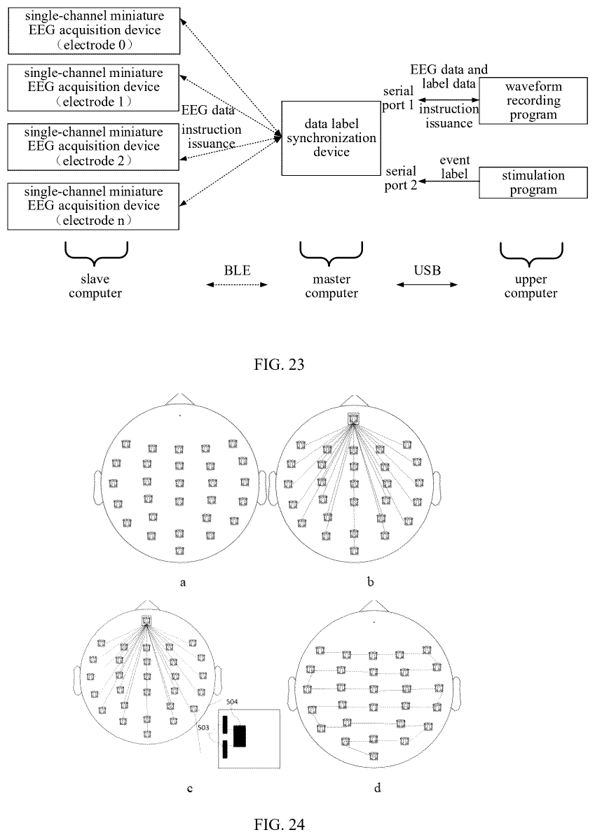

As shown in FIG. 23, the plurality of single-channel miniature EEG acquisition devices acquires EEG data as action electrodes. The plurality of single-channel miniature EEG acquisition devices send the stimulation label data and the EEG data to the data label synchronization device at a certain frequency simultaneously and sequentially. The data label synchronization device sends the data to the waveform recording program of the upper computer for displaying the waveform and the label data. The number of single-channel miniature EEG acquisition devices may be expanded in leads based on the experimental needs, which can be set up to 32 at most.

The single-channel miniature EEG acquisition device 1 is configured to acquire scalp EEG signals, and the number of which may be selected based on the experimental needs for measuring EEG signals, and the number of which is greater than or equal to 2. The stimulation tag data and the EEG data are transmitted to the device (2) for data label synchronization in the BLE way.

In particular, the connection forms of the plurality of multi-channel EEG acquisition devices include an independent electrode, a unipolar lead, an averaged lead, and a bipolar lead. The method of independent electrode acquisition is shown as a in FIG. 24. Each single-channel EEG acquisition device adopts the Laplace dry electrodes with two signal inputs, and each single-channel EEG acquisition device collects one way of EEG signals. The method of unipolar lead acquisition is shown as b in FIG. 24. The electrodes adopt the electrodes with one input signal, and the inverting inputs of all the acquisition devices are connected to one acquisition electrode with no acquisition circuit. Each of the other acquisition device outputs one way of EEG signals. The method of averaged lead acquisition is shown as c in FIG. 24. The electrodes adopt the electrodes with one input signal. The substrate of the PCB of the electrodes with one input signal has a summing resistor to connect the two pads. The inverting inputs of all the acquisition devices are connected to one acquisition electrode with no acquisition circuit, in which the acquisition electrode is not connected to the scalp. Each acquisition device outputs one way of EEG signals. The method of bipolar lead acquisition is shown as d in FIG. 24. The electrodes adopt the electrodes with one input signal. And the inverted input of the former acquisition device is connected to the non-inverting input of the latter acquisition device. And each acquisition device outputs one way of EEG signals.

The device 2 for data label synchronization with dual serial ports based on the BLE is configured to receive the command sent by the waveform recording program 301 of the upper computer 3 and the stimulation label sent by the stimulation program 302 of the upper computer 3 and transmit the command to the single-channel miniature EEG acquisition device; and receive and synchronize the EEG data of a plurality of single-channel miniature EEG acquisition devices 1 and the stimulation label of the stimulus program 302 and transmit the data and label to the waveform recording program 301 of the upper computer 3. The stimulation label may be further described as, e.g., the label information of the different flashing boxes that start flashing when the SSVEP test is performed, and 1, 2, 3 . . . may be configured to represent the label information of which stimulus box starts flashing. The upper computer 3 program includes a waveform recording program 301 and a stimulation program 302, which may be displayed separately by 2 monitors. The waveform recording program 301 is configured for the experimenter to observe the acquired EEG signal waveform, the issuance of the control command, and the storage of the EEG data of the participant. The stimulation program 302 is configured to display a stimulation interface on which the participant is gazing, and the stimulation program 302 sends stimulation label start information to the device 2 for data label synchronization through the serial port 2 before flashing stimulation is started.

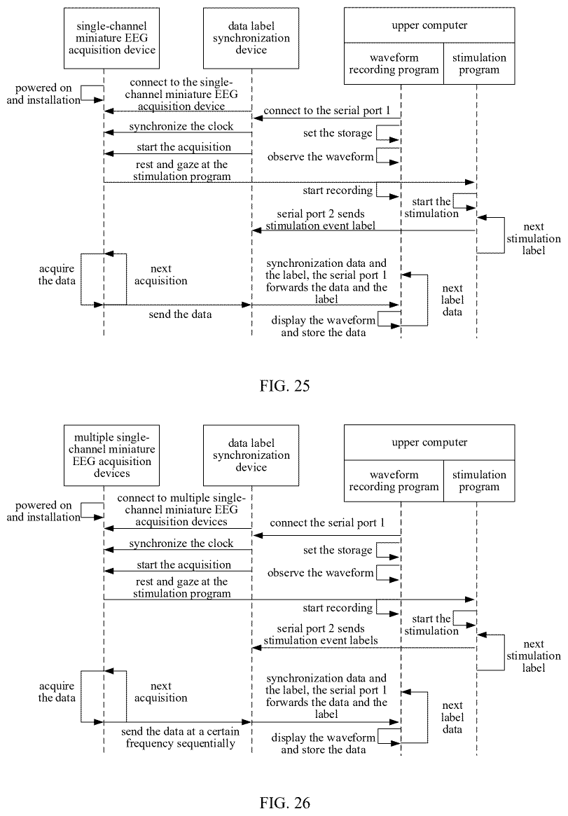

Embodiment 1: the experimental equipment is shown in FIG. 2, and the process is shown in FIG. 25.
1) Power on: the toggle switch 604 is turned on to start the single-channel miniature EEG acquisition device 1.
2) Mounting the device: the device 1 is fixed at the occipital region of the participant by locking the hair through the buckle. The specific steps of wearing the device may be: opening the buckle 906 of the hairpin structure 9 to the two outer sides, putting a certain amount of hair 10 into the groove 904, adjusting the height of the acquisition device 1 by applying downward pressure for a good connection between the electrodes and the scalp; and closing the buckle 906 inwardly to fix the acquisition device 1 to the scalp.
4) (The experimenter) inserts the device 2 for data label synchronization into the USB interface of the computer, the device 2 automatically connects the device 1 and carries out the clock synchronization, and sets up the start acquisition command after the clock synchronization.
5) (The experimenter) sets the waveform recording program 301 on a monitor, selects the serial port 1, connects the device 2 through the serial port 1, and sets and names the data saving path. The EEG waveform displayed by waveform recording program 301 is observed to see whether it is correct.
6) The participant rests for one minute and gazes at the stimulation program 302 interface on the other monitor.
7) (The experimenter) sets the waveform recording program 301 to start recording the data and starts the stimulation program 302.
8) The stimulation program 302 sends the corresponding stimulation label information to the device 2 through the serial port 2 when a certain stimulus block of the stimulation program 302 starts to flash. The stimulation program displays different stimuli according to a specific time and sends different stimulation label data to the device 2.
9) The device 1 sends the data to the device 2 at a certain frequency sequentially. The device 2 synchronizes the data and the label and sends the data and the label to the waveform recording program 301 through the serial port 1. The waveform recording program 301 displays the EEG waveform and stores the data. The device 1 acquires the data cyclically and sends the data to the device 2.

The single-channel miniature EEG acquisition system circuit in the embodiments is miniaturized, portable, comfortable, and stealthy. The acquisition may be hidden between the hairs, which is more acceptable to the user and improves the user experience. The direct connection between the acquisition electrode and the acquisition circuit reduces the possibility of introducing common mode interference or other noise. The design of replaceable electrodes reduces the cost of the experiment.

Embodiment 2: the experimental equipment is shown in FIG. 22, and the process is shown in FIG. 26.
1) Power on: the toggle switch 604 is turned on to start the plurality of single-channel miniature EEG acquisition devices 1.
2) Mounting the device: the plurality of devices 1 are fixed at the occipital region of the participant by locking the hair through the buckle. The position of fixing may be placed according to the 10-20 standard leads, or may be placed according to the experimental needs. The specific steps of wearing the device may be: opening the buckle 906 of the hairpin structure 9 to the two outer sides, putting a certain amount of hair 10 into the groove 904, adjusting the height of the acquisition device 1 by applying downward pressure for a good connection between the electrodes and the scalp; and closing the buckle 906 inwardly to fix the acquisition device 1 to the scalp.
4) (The experimenter) inserts the device 2 for data label synchronization into the USB interface of the computer, the device 2 automatically connects the device 1 and carries out the clock synchronization, and sets up the start acquisition command after the clock synchronization.
5) (The experimenter) sets the waveform recording program 301 on a monitor to select the serial port 1, connects the device 2 through the serial port 1, and sets and names the data saving path. The EEG waveform displayed by waveform recording program 301 is observed to see whether it is correct.
6) The participant rests for one minute and gazes at the stimulation program 302 interface on the other monitor.
7) (The experimenter) sets the waveform recording program 301 to start recording the data and starts the stimulation program 302.
8) The stimulation program 302 sends the corresponding stimulation label information to the device 2 through the serial port 2 when a certain stimulus block of the stimulation program 302 starts to flash. The stimulation program displays different stimulus according to a specific time and sends different stimulation label data to the device 2.
9) The plurality of devices 1 send the data to the device 2 at a certain frequency sequentially. The device 2 synchronizes the data and the label and sends the data and the label to the waveform recording program 301 through the serial port 1. The waveform recording program 301 displays the EEG waveform and stores the data. The device 1 acquires the data cyclically and sends the data to the device 2.

The single-channel miniature EEG acquisition system circuit in the embodiments is miniaturized, portable, comfortable, and stealthy. The acquisition may be hidden between the hairs, which is more acceptable to the user and improves the user experience. The direct connection between the acquisition electrode and the acquisition circuit reduces the possibility of introducing common mode interference or other noise. The design of replaceable electrodes reduces the cost of the experiment. The use of multi-lead may record the EEG information of more areas, which is more comprehensive and accurate for the restoration of the brain intention.

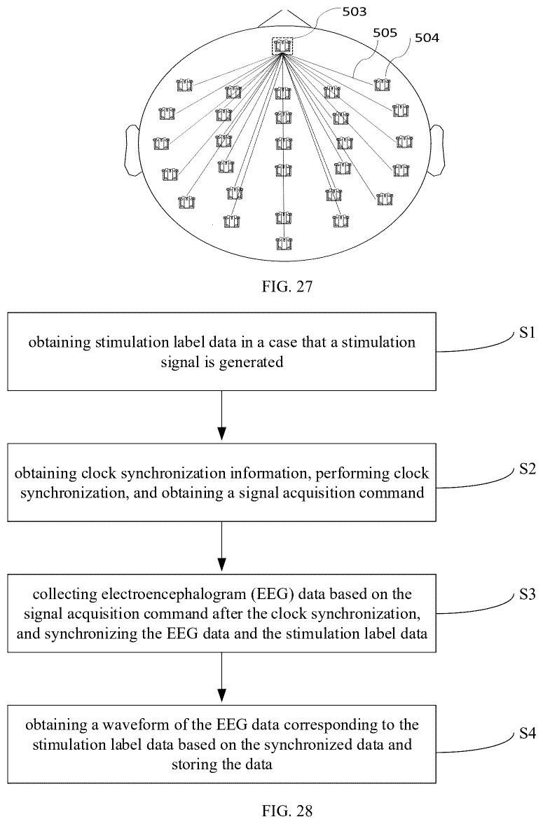

Embodiment 3: the experimental equipment is shown in FIG. 22, and the connection schematic is shown in FIG. 27. An electrode with one signal input is adopted. The electrode leads the inverting inputs of the plurality of single-channel miniature EEG acquisition devices through a short lead and connects together. The electrode is placed on the head position. As a common reference, the electrode does not have an acquisition circuit. The position of the devices may be placed according to the 10-20 standard leads or be placed according to the experimental needs. The process is shown in FIG. 26.
1) Power on: the toggle switch 604 is turned on to start the plurality of single-channel miniature EEG acquisition devices 1.
2) Mounting the device: the plurality of devices 1 are fixed at the occipital region of the participant by locking the hair through the buckle. The position of fixing may be placed according to the 10-20 standard leads, or may be placed according to the experimental needs. The specific steps of wearing the device may be: opening the buckle 906 of the hairpin structure 9 to the two outer sides, putting a certain amount of hair 10 into the groove 904, adjusting the height of the acquisition device 1 by applying downward pressure for a good connection between the electrodes and the scalp; and closing the buckle 906 inwardly to fix the acquisition device 1 to the scalp.
4) (The experimenter) inserts the device 2 for data label synchronization into the USB interface of the computer, the device 2 automatically connects the device 1 and carries out the clock synchronization, and sets up the start acquisition command after the clock synchronization.
5) (The experimenter) sets the waveform recording program 301 on a monitor, selects the serial port 1, connects the device 2 through the serial port 1, and sets and names the data saving path. The EEG waveform displayed by waveform recording program 301 is observed to see whether it is correct.
6) The participant rests for one minute and gazes at the stimulation program 302 interface on the other monitor.
7) (The experimenter) sets the waveform recording program 301 to start recording the data and starts the stimulation program 302.
8) The stimulation program 302 sends the corresponding stimulation label information to the device 2 through the serial port 2 when a certain stimulus block of the stimulation program 302 starts to flash. The stimulation program displays different stimulus according to a specific time and sends different stimulation label data to the device 2.
9) The plurality of devices 1 send the data to the device 2 at a certain frequency sequentially. The device 2 synchronizes the data and the label and sends the data and the label to the waveform recording program 301 through the serial port 1. The waveform recording program 301 displays the EEG waveform and stores the data. The device 1 acquires the data cyclically and sends the data to the device 2.

The single-channel miniature EEG acquisition system circuit in the embodiments is miniaturized, portable, comfortable, and stealthy. The acquisition may be hidden between the hairs, which is more acceptable to the user and improves the user experience. The connection method of direct connection between the acquisition electrode and the acquisition circuit reduces the possibility of introducing common mode interference or other noise. The design of replaceable electrodes reduces the cost of the experiment. The use of multi-lead may record the EEG information of more areas, which is more comprehensive and accurate for the restoration of the brain intention.

Embodiment 4: the embodiment is a multi-channel SSVEP wheelchair control experiment based on LED lights. The EEG acquisition experiment includes a participant, a plurality of single-channel miniature EEG acquisition devices, a data label synchronization device, and a stimulation generation and data processing device. The stimulus generation and data processing device is set on the front of the wheelchair facing the user. The stimulus generation and data processing device is designed based on a field programmable gate array (FPGA), with twelve LEDs with different blinking frequencies evenly distributed at 30° intervals with a diameter of 20 cm to indicate the direction and angle of the advancement, and one LED in the middle to indicates a stop. The FPGA has an online SSVEP classification system for judging the frequency of SSVEP generated by the acquired EEG signals to determine the direction and angle of the advancement, and then control the wheelchair to advance in the judged direction.

The user wears nine action electrodes located in the occipital region and one reference electrode located in the forehead when using the device. The data label synchronization device is connected to the stimulation generation and data processing device through the USB interface. After the system completes the connection and the clock synchronization, the buzzer drops two times as a reminder, and then the LEDs of different frequencies begin to flash. The user gazes at the LEDs in the direction in which the user wishes to advance, and the stimulus generating and data processing device processes the data, determines the direction of the advancement, and controls the wheelchair to move forward.

Further, a motion imagination experiment may be performed to control the wheelchair to advance. For example, the user imagines that the left and right hand movements represent forward and backward commands; the feet movements represent left and right commands; and the tongue movements represent stop commands.

The acquisition device of the EEG acquisition system in the present embodiment is small in size and light in weight, which does not burden the user and allows the user to perform activities outdoors. The system adopts the BLE for data transmission. The low power consumption design allows the user to use the system outdoors for a long time. The communication method of BLE with one master and multiple slaves may perform an expandable multi-lead EEG experiment. The position of the electrode of the disclosure may be placed according to the need. The electrode may be placed in the occipital area for the SSVEP experiment, or on the head area for the motion imagination experiment.

The multi-channel miniature EEG acquisition system according to the embodiments of the disclosure reduces the use of lead wires, which enables the device to be with low power consumption, miniaturization, and portability. The system is easy to wear and reduces the complexity of the acquisition device and improves the accuracy of the labeling process, which provides a higher scalability and reconfigurability.

To implement the above embodiments, as shown in FIG. 28, a data processing method is also provided in the present embodiment. The method includes the following steps S1 to S4.

At S1, stimulation label data is obtained in a case that a stimulation signal is generated.

At S2, clock synchronization information is obtained, clock synchronization is performed, and a signal acquisition command is obtained.

At S3, EEG data is collected based on the signal acquisition command after the clock synchronization, and the EEG data and the stimulation label data are synchronized.

At S4, a waveform of the EEG data corresponding to the stimulation label data is obtained based on the synchronized data, and the data is stored.

The method for data processing according to the embodiment of the disclosure reduces the use of lead wires, which enables the device to be with low power consumption, miniaturization, and portability. The system is easy to wear, reduces the complexity of the acquisition device, and improves the accuracy of the labeling process, which provides a higher scalability and reconfigurability.

It is further considered that in a physiological signal acquisition scene, the communication method of BLE with one master and multiple slaves may also be applied to physiological signal acquisition. For example, physiological signals such as EEG, electrocardiogram (ECG), electromyography (EMG), etc., are collected by different acquisition devices. The data processing needs to ensure the synchronization of the data, i.e., the data of different physiological signals are on the same time axis. In addition, the EEG and the EMG have very high requirements for time accuracy, and the time error should be at the level of ms. In the related art, the time point of the acquisition data of the BLE communication is usually difficult to correspond accurately. Therefore, the data acquisition process of the BLE communication faces the problem of poor data alignment, which affects the accuracy of physiological signal acquisition. Thus, the problem of low accuracy of data alignment exists in the related art.

The method for data processing according to the embodiments of the disclosure may further includes the following steps: selecting, through the data label synchronization device, a baseline single-channel EEG acquisition device from the plurality of single-channel EEG acquisition devices based on an interrupt time interval corresponding to a monitored radio frequency mapping interrupt event, traversing the plurality of single-channel EEG acquisition devices individually, and when traversing each target single-channel EEG acquisition device among the plurality of single-channel EEG acquisition devices, sending a start acquisition command to the target single-channel EEG acquisition device to cause each target single-channel EEG acquisition device to perform acquisition at a radio frequency mapping interrupt of the target single-channel EEG acquisition device based on a target delay time period corresponding to the target single-channel EEG acquisition device, in which the target delay time period is included in the received start acquisition command, to start a data acquisition at a target acquisition moment of a current first baseline connection interval of the baseline single-channel EEG acquisition device to obtain a plurality of target acquisition data of the target single-channel EEG acquisition device with an acquisition duration being the baseline connection interval individually, and each target single-channel EEG acquisition device performs data transmission at a radio frequency mapping interrupt of the target single-channel EEG acquisition device based on the corresponding target delay time period, to send obtained target acquisition data at a start moment of a next baseline connection interval of the target single-channel EEG acquisition device when obtaining each target acquisition data. The method, which provides a data synchronization mechanism for BLE communication, sets a plurality of slave devices to start acquisition synchronously through the radio frequency mapping interrupt, and sets the plurality of slave devices to send data synchronously, so that the data sent by the plurality of slave devices are at the same moment. The method improves the data quality of the physiological signal acquisition and may improve the accuracy of the data alignment of the BLE communication.

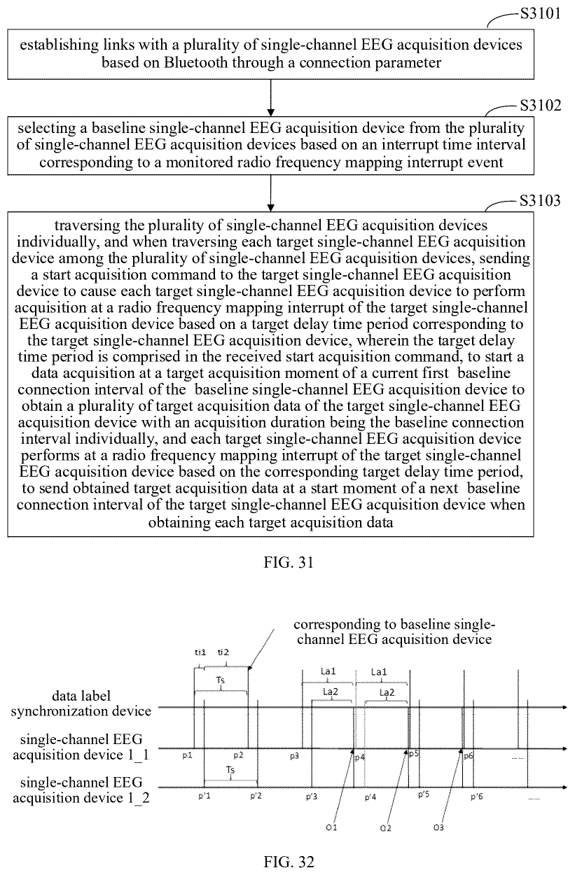

The method for data processing provided in the embodiments of the disclosure is further explained and illustrated below. As shown in FIG. 31, a method for data processing is provided in an embodiment of the disclosure. The method is applied to the data label synchronization device, and further includes the following steps S3101 to S3103.

At step S3101, links with the plurality of single-channel EEG acquisition devices are established based on Bluetooth through a connection parameter.

A connection period of each target link in the links is a baseline connection interval, and an interval between start moments of any two adjacent target links is a connection time threshold; in which the connection parameter includes a number of slave devices, and the baseline connection interval and the connection time threshold.

In the embodiments of the disclosure, the data label synchronization device may be referred to as the master computer or the master device; the single-channel EEG acquisition devices may be referred to as the slave computers or the slave devices. The master device may establish links with a plurality of slave devices based on Bluetooth through a connection parameter. For example, the n single-channel EEG acquisition devices may include a single-channel EEG acquisition device 1_1, a single-channel EEG acquisition device 1_2, . . . , and a single-channel EEG acquisition device 1_n. In embodiments of the disclosure, the number of single-channel EEG acquisition devices is multiple, and the disclosure does not specifically limit the exact number of the single-channel EEG acquisition devices. It may be understood that in some embodiments of the disclosure, the data label synchronization device may also be arranged on the terminal device, i.e., the data label synchronization device may be a device that is integrated with the terminal device.

In order to more succinctly illustrate the data synchronization of the data processing method of the embodiments of the disclosure, the following embodiments are all illustrated as examples of the plurality of single-channel EEG acquisition devices that establish links with the data label synchronization device based on Bluetooth are the single-channel EEG acquisition device 1_1 and the single-channel EEG acquisition device 1_2. The connection period of each target link in the links is a baseline connection interval Ts, and the interval between the start moments of any two adjacent target links is a connection time threshold time 01.

At step S3102, a baseline single-channel EEG acquisition device is selected from the plurality of single-channel EEG acquisition devices based on an interrupt time interval corresponding to a monitored radio frequency mapping interrupt event.

The interrupt time interval represents a time interval between a current radio frequency mapping interrupt event and a previous radio frequency mapping interrupt event; in which the baseline single-channel EEG acquisition device is a single-channel EEG acquisition device that first establishes a target link with the data label synchronization device during the baseline connection interval.

In a specific implementation, the data label synchronization device communicates with the plurality of single-channel EEG acquisition devices through the BLE. After the data label synchronization device establishes links with the plurality of single-channel EEG acquisition devices based on Bluetooth through a connection parameter, the data label synchronization device connects with each of the plurality of single-channel EEG acquisition devices once during any of the baseline connection interval of the data label synchronization device. The baseline single-channel EEG acquisition device is a single-channel EEG acquisition device that first establishes the target link with the data label synchronization device during the baseline connection interval of the data label synchronization device. In an embodiment of the disclosure, a radio frequency mapping interrupt event refers to an external interrupt of the mapping IO that is triggered when the master device performing transmission or reception.

In an optional embodiment, the process of selecting a baseline single-channel EEG acquisition device from the plurality of single-channel EEG acquisition devices based on an interrupt time interval corresponding to a monitored radio frequency mapping interrupt event may be realized as follows: in a case that a monitored current interrupt time interval satisfies a preset selection condition, determining a single-channel EEG acquisition device corresponding to the current interrupt time interval as the baseline single-channel EEG acquisition device.

In an embodiment of the disclosure, the preset selection condition is that the current interrupt time interval is greater than the connection time threshold.

In some embodiments, the connection time threshold time 01 may be a value of 5 ms.

For example, if the connection time threshold time 01 is 5 ms, and the current monitored interrupt time interval is determined to be greater than the connection time threshold of 5 ms, the single-channel EEG acquisition device corresponding to the current interrupt time interval is determined as the baseline single-channel EEG acquisition device. Referring to FIG. 32, the interrupt time interval corresponding to the monitored radio frequency mapping interrupt event may be time ti1 and time ti2. If the monitored interrupt time interval is time ti2, and the current monitored interrupt time interval is determined to be greater than the connection time threshold of 5 ms, the single-channel EEG acquisition device 1_1 corresponding to time ti2 is determined as the baseline single-channel EEG acquisition device.

At step S3103, the plurality of single-channel EEG acquisition devices are traversed individually, and when traversing each target single-channel EEG acquisition device among the plurality of single-channel EEG acquisition devices, a start acquisition command is sent to the target single-channel EEG acquisition device to cause each target single-channel EEG acquisition device to perform acquisition at a radio frequency mapping interrupt of the target single-channel EEG acquisition device based on a target delay time period corresponding to the target single-channel EEG acquisition device, in which the target delay time period is included in the received start acquisition command, to start a data acquisition at a target acquisition moment of a current first baseline connection interval of the baseline single-channel EEG acquisition device to obtain a plurality of target acquisition data of the target single-channel EEG acquisition device with an acquisition duration being the baseline connection interval individually, and each target single-channel EEG acquisition device performs data transmission at a radio frequency mapping interrupt of the target single-channel EEG acquisition device based on the corresponding target delay time period, to send obtained target acquisition data at a start moment of a next baseline connection interval of the target single-channel EEG acquisition device when obtaining each target acquisition data.

The target acquisition moment is a moment preceding a start moment of a next baseline connection interval of the baseline single-channel EEG acquisition device with a time interval of a preset acquisition time threshold, and the target acquisition data includes a same number of data points.

In a specific implementation, the start acquisition command is sent to all single-channel EEG acquisition devices in sequence. The formula for calculating the acquisition delay is: acquisition delay=baseline connection interval −5*i−1, in which i is the sequence identify of the slave devices, and the number of slave devices >i≥0.

For example, with reference to FIG. 32, a target delay time period corresponding to the single-channel EEG acquisition device 1_1 is La1, a target delay time period corresponding to the single-channel EEG acquisition device 1_2 is La2. The plurality of single-channel EEG acquisition devices are traversed individually. When traversing each target single-channel EEG acquisition device among the plurality of single-channel EEG acquisition devices, for example, the single-channel EEG acquisition device 1_1, a start acquisition command is sent to the target single-channel EEG acquisition device 1_1, to cause the single-channel EEG acquisition device 1_1 to perform acquisition at a radio frequency mapping interrupt p3 of the single-channel EEG acquisition device 1_1 based on the target delay time period La1 corresponding to the target single-channel EEG acquisition device 1_1, in which the target delay time period is included in the received start acquisition command, to start a data acquisition at a target acquisition moment O1 of a current first baseline connection interval (a time period between p3 and p4) of the single-channel EEG acquisition device 1_1, to obtain a plurality of target acquisition data of the single-channel EEG acquisition device 1_1 with an acquisition duration Ts being the baseline connection interval individually. The single-channel EEG acquisition device 1_1 performs data transmission at a radio frequency mapping interrupt p4 of the single-channel EEG acquisition device 1_1 based on the corresponding target delay time period La1. In this way, the obtained target acquisition data is sent at a start moment of a next baseline connection interval of the single-channel EEG acquisition device 1_1 when obtaining each target acquisition data. For example, when obtaining one target acquisition data in the time period between p4 and p5, the obtained target acquisition data is sent at a start moment of a next baseline connection interval (a time period between the p5 and p6) of the single-channel EEG acquisition device 1_1. The target acquisition moment O1 is a moment preceding a start moment of a next baseline connection interval of the baseline single-channel EEG acquisition device with a time interval of a preset acquisition time threshold. The target acquisition data includes a same number of data points. The acquisition time threshold may be 1 ms. The start acquisition command is sent to the single-channel EEG acquisition device 1_2. The data acquisition and sending process of the single-channel EEG acquisition device 1_2 is similar to the data acquisition and sending process of the single-channel EEG acquisition device 1_1 as described in the preceding section, and the similarities will not be further described. Thus, the single-channel EEG acquisition device 1_1 and the single-channel EEG acquisition device 1_2 start data acquisition synchronously at the target acquisition moment O1. The target acquisition data obtained by the single-channel EEG acquisition device 1_1 and the single-channel EEG acquisition device 1_2 in the same baseline connection interval may be sent synchronously at the starting moment of the next baseline connection interval.

In an optional embodiment, at step S3103, after sending the start acquisition command to the target data label synchronization device, target information is obtained by packing target acquisition data sent by the plurality of data label synchronization devices and received in a same baseline connection interval.

The method of the above embodiments may further provides a data synchronization mechanism for BLE communication, in which a plurality of slave devices are set to start acquisition synchronously through the radio frequency mapping interrupt, and the plurality of slave devices are set to send data synchronously, so that the data sent by the slave devices are at the same moment. The method improves the data quality of the physiological signal acquisition and may improve the accuracy of the data alignment of the BLE communication and the accuracy of data synchronization.

Another method for data processing, applied to a master device is provided in an embodiment of the disclosure. The method further includes the following steps A1 to A8.

At step A1, links with the plurality of single-channel EEG acquisition devices are established based on Bluetooth through a connection parameter.

A connection period of each target link in the links is a baseline connection interval, and an interval between start moments of any two adjacent target links is a connection time threshold; in which the connection parameter includes a number of slave devices, and the baseline connection interval and the connection time threshold.

In an embodiment of the disclosure, the number of the slave devices may also be referred to as a count of the slave devices.

At step A2, a baseline single-channel EEG acquisition device is selected from the plurality of single-channel EEG acquisition devices based on an interrupt time interval corresponding to a monitored radio frequency mapping interrupt event.

The interrupt time interval represents a time interval between a current radio frequency mapping interrupt event and a previous radio frequency mapping interrupt event; in which the baseline single-channel EEG acquisition device is a single-channel EEG acquisition device that first establishes a target link with the data label synchronization device during the baseline connection interval.

At step A3, the plurality of single-channel EEG acquisition devices are traversed individually, and when traversing each target single-channel EEG acquisition device among the plurality of single-channel EEG acquisition devices, a start acquisition command is sent to the target single-channel EEG acquisition device to cause each target single-channel EEG acquisition device to perform acquisition at a radio frequency mapping interrupt of the target single-channel EEG acquisition device based on a target delay time period corresponding to the target single-channel EEG acquisition device, in which the target delay time period is included in the received start acquisition command, to start a data acquisition at a target acquisition moment of a current first baseline connection interval of the baseline single-channel EEG acquisition device to obtain a plurality of target acquisition data of the target single-channel EEG acquisition device with an acquisition duration being the baseline connection interval individually, and each target single-channel EEG acquisition device performs data transmission at a radio frequency mapping interrupt of the target single-channel EEG acquisition device based on the corresponding target delay time period, to send obtained target acquisition data at a start moment of a next baseline connection interval of the target single-channel EEG acquisition device when obtaining each target acquisition data.

The target acquisition moment is a moment preceding a start moment of a next baseline connection interval of the baseline single-channel EEG acquisition device with a time interval of a preset acquisition time threshold, and the target acquisition data includes a same number of data points. The target acquisition data includes target acquisition information and a sending time point corresponding to the target acquisition information. The sending time point represents an end moment of an acquisition time duration corresponding to the target acquisition information.

At step A4, if a target stimulation label sent by an upper computer is received, the operations illustrated at steps A5 to A7 are performed.

At step A5, a first time point of a first alignment timer of the data label synchronization device is obtained in a case of receiving the target stimulation label.

At step A6, a first sending time point included in the first target acquisition data is obtained based on first target acquisition data; in which the first target acquisition data is first received target acquisition data sent by the baseline single-channel EEG acquisition device after receiving the target stimulation label; in which the first sending time point is a time point of a second alignment timer of the baseline single-channel EEG acquisition device at the end of an acquisition duration corresponding to the target acquisition information; in which the second alignment timer is a timer that is synchronized with the first alignment timer.

At step A7, label position identification information of the target stimulation label is obtained based on the first sending time point and the first time point.

In a specific implementation, the process of obtaining the label position identification information of the target stimulation label based on the first sending time point and the first time point, may include obtaining the label position identification information of the target stimulation label by a following formula:

$$offset = M - ( t 1 - t 2 ) / ( 1000 / f )$$

The offset is the label position identification information of the target stimulation label, configured to determine the target acquisition data corresponding to the target stimulation label, a value range of the offset is 0<offset≤2*M, it is determined that the target stimulation label corresponds to first target acquisition data in a case that 0<offset≤M, and it is determined that the target stimulation label corresponds to second target acquisition data in a case M<offset≤2M, in which the second target acquisition data is next target acquisition data of the first target acquisition data sent by the baseline single-channel EEG acquisition device.

The “t1” is the first sending time point.

The “t2” is the first time point that represents a time point when the data label synchronization device receives a stimulation label sent by a stimulation program of the upper computer.

The “M” is a number of data points included in each target acquisition data.

The “f” is a sampling rate.

At step A8, the target information is obtained by packing target acquisition data sent by the plurality of single-channel EEG acquisition devices and received in a same baseline connection interval based on the target stimulation label.

In an optional embodiment, the process of obtaining the target information by packing target acquisition data sent by the plurality of single-channel EEG acquisition devices and received in a same baseline connection interval based on the target stimulation label is implemented by the following steps B1 to B2.

At step B1, in a case of receiving the target stimulation label, the target stimulation label, the label position identification information and target acquisition data sent by the plurality of single-channel EEG acquisition devices and received in a first baseline connection interval are packed.

At step B2, in a case of not receiving the target stimulation label, a preset empty label, preset empty position identification information and the target acquisition data sent by the plurality of single-channel EEG acquisition devices and received in the first baseline connection interval are packed.

In an optional embodiment, after selecting the baseline single-channel EEG acquisition device from the single-channel EEG acquisition devices based on the interrupt time interval corresponding to the monitored radio frequency mapping interrupt event at step A2, and before traversing the plurality of single-channel EEG acquisition devices individually, and when traversing each target single-channel EEG acquisition device among the plurality of single-channel EEG acquisition devices, sending a start acquisition command to the target single-channel EEG acquisition device at step A3, the following steps C1 to C3 are further included.

At step C1, a first current time point of the first alignment timer is read.

At step C2, a first alignment reference time point is obtained by summing the first current time point with the baseline connection interval.

Step C3, the first alignment reference time point is sent to the baseline single-channel EEG acquisition device to cause the baseline single-channel EEG acquisition device to realize time synchronization between the second alignment timer and the first alignment timer by determining the first alignment reference time point as a current time point of the second alignment timer.

In an optional embodiment, after sending the first alignment reference time point to the baseline single-channel EEG acquisition device at step C3, the following steps D1 to D4 are further included.

At step D1, in a case that an interval between the current time point and a time point of sending the first alignment reference time point to the baseline single-channel EEG acquisition device is monitored to be increased by a preset calibration interval threshold, the following operations of step D2 to step D4 are performed.

At step D2, a second current time of the first alignment timer is read.

At step D3, a second alignment reference time point is obtained by summing the second current time point with the baseline connection interval.

At step D4, the second alignment reference time point is sent to the baseline single-channel EEG acquisition device to cause the baseline single-channel EEG acquisition device to calibrate the current time point of the second alignment timer based on the second alignment reference time point.

The methods in the embodiments further implement a mechanism for determining the position of the data acquisition of the slave device corresponding to the stimulation event, and realizes the alignment synchronization of the stimulation event and the data acquisition of the slave device. The method improves the accuracy of the data synchronization and the accuracy of the label synchronization, which may further improve the accuracy of the physiological signal acquisition and improve the efficiency of the data synchronization of the BLE communication.

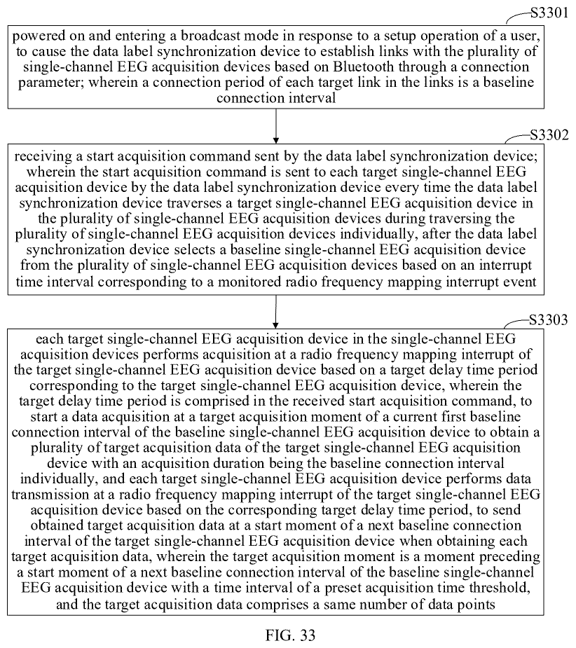

As shown in FIG. 33, another method for data processing is provided in an embodiment of the disclosure. The method is applied to the single-channel EEG acquisition device, and further includes the following steps S3101 to S3103.

At step S3301, the device is powered on and enters a broadcast mode in response to a setup operation of a user, to cause the data label synchronization device to establish links with the plurality of single-channel EEG acquisition devices based on Bluetooth through a connection parameter; in which a connection period of each target link in the links is a baseline connection interval.

At step S3302, a start acquisition command sent by the data label synchronization device is received. The start acquisition command is sent to each target single-channel EEG acquisition device by the data label synchronization device every time the data label synchronization device traverses a target single-channel EEG acquisition device in the plurality of single-channel EEG acquisition devices during traversing the plurality of single-channel EEG acquisition devices individually, after the data label synchronization device selects a baseline single-channel EEG acquisition device from the plurality of single-channel EEG acquisition devices based on an interrupt time interval corresponding to a monitored radio frequency mapping interrupt event.

At step S3303, each target single-channel EEG acquisition device in the single-channel EEG acquisition devices performs acquisition at a radio frequency mapping interrupt of the target single-channel EEG acquisition device based on a target delay time period corresponding to the target single-channel EEG acquisition device, in which the target delay time period is included in the received start acquisition command, to start a data acquisition at a target acquisition moment of a current first baseline connection interval of the baseline single-channel EEG acquisition device to obtain a plurality of target acquisition data of the target single-channel EEG acquisition device with an acquisition duration being the baseline connection interval individually, and each target single-channel EEG acquisition device performs data transmission at a radio frequency mapping interrupt of the target single-channel EEG acquisition device based on the corresponding target delay time period, to send obtained target acquisition data at a start moment of a next baseline connection interval of the target single-channel EEG acquisition device when obtaining each target acquisition data, in which the target acquisition moment is a moment preceding a start moment of a next baseline connection interval of the baseline single-channel EEG acquisition device with a time interval of a preset acquisition time threshold, and the target acquisition data includes a same number of data points.

In an optional embodiment, after powering on and entering the broadcast mode in response to the setup operation of the user at step S3301, and before receiving the start acquisition command sent by the data label synchronization device at S3302, in a case that the single-channel EEG acquisition device receives a first alignment reference time point sent by the data label synchronization device, the first alignment reference time point is determined as a current time point of a second alignment timer, to realize time synchronization between the second alignment timer and the first alignment timer, in which the first alignment reference time point is obtained by summing a first current time point with the baseline connection interval, and the first current time point is read after selecting the baseline single-channel EEG acquisition device from the single-channel EEG acquisition devices based on the interrupt time interval corresponding to the monitored radio frequency mapping interrupt event, and before traversing the single-channel EEG acquisition devices individually and sending the start acquisition command to the target single-channel EEG acquisition device for each target single-channel EEG acquisition device among the single-channel EEG acquisition devices traversed among the single-channel EEG acquisition devices.

In an optional embodiment, after powering on and entering the broadcast mode in response to the setup operation of the user at step S3301, and before receiving the start acquisition command sent by the data label synchronization device at S3302, the method further includes the following steps E1 to E2.

At step E1, in a case that the single-channel EEG acquisition device receives a first alignment reference time point sent by the data label synchronization device, the first alignment reference time point is determined as a current time point of a second alignment timer, to realize time synchronization between the second alignment timer and the first alignment timer.

The first alignment reference time point is obtained by summing a first current time point with the baseline connection interval, and the first current time point is read after selecting the baseline single-channel EEG acquisition device from the single-channel EEG acquisition devices based on the interrupt time interval corresponding to the monitored radio frequency mapping interrupt event, and before traversing the single-channel EEG acquisition devices individually and sending the start acquisition command to the target single-channel EEG acquisition device for each target single-channel EEG acquisition device among the single-channel EEG acquisition devices traversed among the single-channel EEG acquisition devices.

At step E2, in a case that the single-channel EEG acquisition device receives a second alignment reference time point sent by the data label synchronization device and determines that a difference between the second alignment reference time point and the current time point of the second alignment timer is not greater than the baseline connection interval, the current time point of the second alignment timer is replaced with the second alignment reference time point.

The second alignment reference time point is sent to the baseline single-channel EEG acquisition device by the data label synchronization device, and obtained by summing a second current time point of the first alignment timer with the baseline connection interval, in which the second current time point is read by the data label synchronization device in a case that an interval between the current time point and a time point of sending the first alignment reference time point to the baseline single-channel EEG acquisition device is monitored to be increased by a preset calibration interval threshold.

The method for data processing provided in the above embodiments further provides a data synchronization mechanism for BLE communication, a plurality of slave devices are set to start acquisition synchronously based on the radio frequency mapping interrupt, and the plurality of slave devices are set to send data synchronously, which ensures that each of the slave devices sent data at the same moment. The method improves the accuracy of physiological signal acquisition, which may improve the accuracy of the data alignment of the BLE communication and the accuracy of data synchronization.

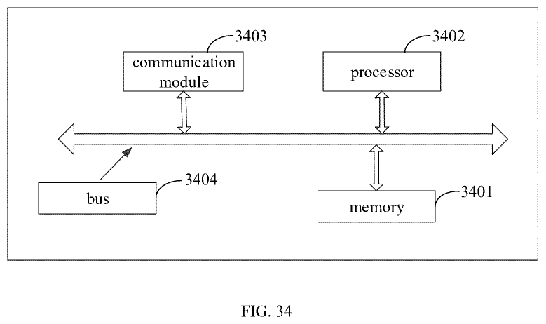

Based on the same inventive concept as the method embodiments described above, an electronic device is also provided in an embodiment of the disclosure. The electronic device may be used in a process of data processing in a multi-channel miniature EEG acquisition system. In some embodiments, the electronic device may be applied to a data label synchronization device of the BLE. In some other embodiments, the electronic device may be applied to a single-channel EEG acquisition device of the BLE. In an embodiment, as shown in FIG. 34, the structure of the electronic device may include a memory 3401, a communication module 3403, and one or more processors 3402.

The memory 3401 is configured to store an embedded program executed by the processor 3402. The memory 3401 may primarily include a storage program area and a storage data area. The storage program area may store an operating system, and programs required to run the instant messaging function, etc. The storage data area may store various instant messaging information and operation instruction sets, etc.

The memory 3401 may be a volatile memory, such as a random-access memory (RAM), or a non-volatile memory. Alternatively, the memory 3401 is any other medium capable of being configured to carry or store desired program code in the form of instructions or data structures and capable of being accessed by a computer, but is not limited to this. The memory 3401 may be a combination of the above memories.

The processor 3402 may include one or more central processing units (CPUs) or the processor 3402 may be a digital processing unit, etc. The processor 3402 is configured to achieve data synchronization for data processing of any of the above embodiments when calling the embedded program stored in the memory 3401.

The communication module 3403 is configured to communicate with a terminal device or other servers.

The embodiments of the disclosure do not limit the specific connection medium between the memory 3401, the communication module 3403 and the processor 3402. As shown in FIG. 34, in an embodiment of the disclosure, the memory 3401 and the processor 3402 are connected through a bus 3404, which is indicated by a thick line in FIG. 34, and the connection between other components is only for schematic illustration and is not to be cited as a limitation. The bus 3404 may be an address bus, a data bus, a control bus, and the like. For ease of representation, the buses are represented in FIG. 34 by only one thick line, but this does not mean that there is only one bus or one type of bus.

The multi-channel miniature EEG acquisition system and method and the electronic device provided in embodiments of the disclosure design a method of combining a hairpin structure with gel fixing based on the single-channel miniature EEG acquisition devices, so that the miniature EEG acquisition device may be fixed on the scalp through the hairpin structure design of the device, which is used together with viscous gel electrodes, to enable the electrodes and the scalp to become viscous. The use of the two fixing methods, namely the hairpin and the auxiliary electrodes, have the characteristics of miniaturization, stealthiness, and strong wearability, which are more easily accepted by the consumers. The data label synchronization device based on dual serial ports reduces the complexity of the acquisition device. The experimental scene is not limited to the laboratory and is more suitable for outdoor use by synchronizing the EEG data and the label information through the data label synchronization device, which has the characteristics of low power consumption, portability, miniaturization, and stealthiness. It is more acceptable to the users and improves the user experience. The multi-channel EEG acquisition system utilizes the communication method of BLE with one master and multiple slaves, in which a plurality of single-channel miniature EEG acquisition devices collect EEG data, the number and the positions of the devices may be set according to the experimental needs, and the connection method may be designed according to the experimental needs, which provides a higher scalability and reconfigurability.

In the description, terms such as “an embodiment,” “some embodiments,” “an example,” “a specific example,” or “some examples,” means that a particular feature, structure, material, or characteristic described in combination with the embodiment or example is included in at least one embodiment or example of the disclosure. Thus, the appearances of the above terms in various places throughout this specification are not necessarily referring to the same embodiment or example of the disclosure. Furthermore, the particular features, structures, materials, or characteristics may be combined in any suitable manner in one or more embodiments or examples. In addition, without contradicting each other, those skilled in the art may combine and combine different embodiments or examples and features of different embodiments or examples described in this specification.

It may be understood that although embodiments of the disclosure have been shown and described above, the above embodiments are exemplary and are not to be construed as a limitation of the disclosure, and those skilled in the art may make changes, modifications, substitutions, and variations of the above embodiments within the scope of the disclosure.

1. A multi-channel miniature electroencephalogram (EEG) acquisition system, comprising: a plurality of single-channel EEG acquisition devices, a data label synchronization device, and a terminal device, wherein the terminal device comprises a waveform recording module and a stimulation module;
the stimulation module is configured to transmit stimulation label data to the data label synchronization device through a universal serial bus (USB) interface in a case that a stimulation signal is generated;
the data label synchronization device is configured to issue clock synchronization information and a control command, and receive the stimulation label data;
the plurality of single-channel EEG acquisition devices are configured to receive the clock synchronization information, collect EEG data based on the control command, and transmit the EEG data to the data label synchronization device through Bluetooth low energy (BLE);
the data label synchronization device is further configured to synchronize the EEG data and the stimulation label data, and transmit synchronized data and the stimulation label data to the waveform recording module; and
the waveform recording module is configured to display a waveform of the EEG data corresponding to the stimulation label data and perform data store.

2. The system according to claim 1, wherein each single-channel EEG acquisition device comprises an acquisition circuit, wherein the acquisition circuit comprises: a first Microcontroller Unit (MCU) and wireless transmission module, a signal acquisition module, a first power supply module, and a battery module, wherein the signal acquisition module comprises an acquisition circuit board, the acquisition circuit board is configured to collect an EEG signal, and the signal acquisition module communicates with the first MCU and wireless transmission module through a serial peripheral interface (SPI), the first MCU and wireless transmission module is configured to read the EEG signal collected by the signal acquisition module and transmit the EEG signal to the data label synchronization device through the BLE, the first power supply module is arranged on a power supply board, wherein the first power supply module comprises a voltage converter and a circuit of the voltage converter, the voltage converter converts a battery voltage of the battery module into a positive voltage and a negative voltage, to supply power to the signal acquisition module and the first MCU and wireless transmission module, and the battery module is connected to the first power supply module.

3. The system according to claim 1, wherein the data label synchronization device comprises a second MCU and wireless transmission module, a serial port module and a second power supply module, wherein the second MCU and wireless transmission module is configured to receive and synchronize EEG data collected by the plurality of single-channel EEG acquisition devices, the serial port module is connected to the second MCU and wireless transmission module and the terminal device, the serial port module comprises a first serial port circuit and a second serial port circuit, wherein the first serial port circuit communicates with the waveform recording module of the terminal device, the first serial port circuit is configured to forward the EEG data and the acquisition command synchronized by the second MCU and wireless transmission module, the second serial port circuit communicates with the stimulation module of the terminal device, the second serial port circuit is configured to receive the stimulation label data, and the second power supply module is configured to supply power to the second MCU and wireless transmission module and the serial port module.

4. (canceled)

5. The system according to claim 1, wherein the data label synchronization device is configured to:
establish links with the plurality of single-channel EEG acquisition devices based on Bluetooth through a connection parameter; wherein a connection period of each target link in the links is a baseline connection interval, and an interval between start moments of any two adjacent target links is a connection time threshold; wherein the connection parameter comprises a number of slave devices, and the baseline connection interval and the connection time threshold;
select a baseline single-channel EEG acquisition device from the plurality of single-channel EEG acquisition devices based on an interrupt time interval corresponding to a monitored radio frequency mapping interrupt event; wherein the interrupt time interval represents a time interval between a current radio frequency mapping interrupt event and a previous radio frequency mapping interrupt event; wherein the baseline single-channel EEG acquisition device is a single-channel EEG acquisition device that first establishes a target link with the data label synchronization device during the baseline connection interval; and
traverse the plurality of single-channel EEG acquisition devices individually, and when traversing each target single-channel EEG acquisition device among the plurality of single-channel EEG acquisition devices, send a start collection command to the target single-channel EEG acquisition device, wherein each target single-channel EEG acquisition device is configured to perform acquisition at a radio frequency mapping interrupt of the target single-channel EEG acquisition device based on a target delay time period corresponding to the target single-channel EEG acquisition device, wherein the target delay time period is comprised in the received start acquisition command, to start a data acquisition at a target acquisition moment of a current first baseline connection interval of the baseline single-channel EEG acquisition device to obtain a plurality of target acquisition data of the target single-channel EEG acquisition device with an acquisition duration being the baseline connection interval individually, and each target single-channel EEG acquisition device performs data transmission at a radio frequency mapping interrupt of the target single-channel EEG acquisition device based on the corresponding target delay time period, to send obtained target acquisition data at a start moment of a next baseline connection interval of the target single-channel EEG acquisition device when obtaining each target acquisition data, wherein the target acquisition moment is a moment preceding a start moment of a next baseline connection interval of the baseline single-channel EEG acquisition device with a time interval of a preset acquisition time threshold, and the target acquisition data comprises a same number of data points.

6. The system according to claim 5, wherein the data label synchronization device is configured to:
in a case that a monitored current interrupt time interval satisfies a preset selection condition, determine a single-channel EEG acquisition device corresponding to the current interrupt time interval as the baseline single-channel EEG acquisition device, wherein the selection condition is that the current interrupt time interval is greater than the connection time threshold.

7. The system according to claim 5, wherein the data label synchronization device is further configured to:
after sending the start acquisition command to the target data label synchronization device, obtain target information by packing target acquisition data sent by the plurality of data label synchronization devices and received in a same baseline connection interval.

8. The system according to claim 7, wherein the target acquisition data comprises target acquisition information and a sending time point corresponding to the target acquisition information; wherein the sending time point represents an end moment of an acquisition duration corresponding to the target acquisition information; wherein the data label synchronization device is further configured to:
in a case of receiving a target stimulation label from the terminal device after sending the start acquisition command to the target data label synchronization device and before packing the target acquisition data sent by the plurality of data label synchronization devices and received in the same baseline connection interval, perform following operations:
obtaining a first time point of a first alignment timer of the data label synchronization device in a case of receiving the target stimulation label;
obtaining, based on first target acquisition data, a first sending time point comprised in the first target acquisition data; wherein the first target acquisition data is first received target acquisition data sent by the baseline single-channel EEG acquisition device after receiving the target stimulation label; wherein the first sending time point is a time point of a second alignment timer of the baseline single-channel EEG acquisition device at the end of an acquisition duration corresponding to the first target acquisition information; wherein the second alignment timer is a timer that is synchronized with the first alignment timer; and
obtaining label position identification information of the target stimulation label by a following formula:
$$offset = M - ( t 1 - t 2 ) / ( 1000 / f )$$
wherein the offset is the label position identification information of the target stimulation label, configured to determine the target acquisition data corresponding to the target stimulation label, a value range of the offset is 0<offset≤2*M, it is determined that the target stimulation label corresponds to first target acquisition data in a case that 0<offset≤M, and it is determined that the target stimulation label corresponds to second target acquisition data in a case M<offset≤2M, wherein the second target acquisition data is next target acquisition data of the first target acquisition data sent by the baseline single-channel EEG acquisition device;
“t1” is the first sending time point;
“t2” is the first time point that represents a time point when the data label synchronization device receives a stimulation label sent by a stimulation program of the terminal device;
“M” is a number of data points comprised in each target acquisition data; and
“f” is a sampling rate;
wherein the data label synchronization device is specifically configured to:
in a case of receiving the target stimulation label, pack the target stimulation label, the label position identification information and target acquisition data sent by the plurality of single-channel EEG acquisition devices and received in a first baseline connection interval; and
in a case of not receiving the target stimulation label, pack a preset empty label, preset empty position identification information and the target acquisition data sent by the plurality of single-channel EEG acquisition devices and received in the first baseline connection interval.

9. The system according to claim 8, wherein the data label synchronization device is further configured to, after selecting the baseline single-channel EEG acquisition device from the plurality of single-channel EEG acquisition devices based on the interrupt time interval corresponding to the monitored radio frequency mapping interrupt event, and before traversing the plurality of single-channel EEG acquisition devices individually and sending the start acquisition command to the target single-channel EEG acquisition device for each target single-channel EEG acquisition device among the plurality of single-channel EEG acquisition devices, perform following operations:
reading a first current time point of the first alignment timer;
obtaining a first alignment reference time point by summing the first current time point with the baseline connection interval; and
sending the first alignment reference time point to the baseline single-channel EEG acquisition device, wherein the baseline single-channel EEG acquisition device is configured to realize time synchronization between the second alignment timer and the first alignment timer by determining the first alignment reference time point as a current time point of the second alignment timer.

10. The system according to claim 9, wherein the data label synchronization device is further configured to:
after sending the first alignment reference time point to the baseline single-channel EEG acquisition device, in a case that an interval between the current time point and a time point of sending the first alignment reference time point to the baseline single-channel EEG acquisition device is monitored to be increased by a preset calibration interval threshold, perform following operations:
reading a second current time point of the first alignment timer;
obtaining a second alignment reference time point by summing the second current time point with the baseline connection interval; and
sending the second alignment reference time point to the baseline single-channel EEG acquisition device, wherein the baseline single-channel EEG acquisition device is configured to calibrate the current time point of the second alignment timer based on the second alignment reference time point.

11. The system according to claim 1, wherein each single-channel EEG acquisition device is further configured to:
power on and enter a broadcast mode in response to a setup operation of a user, wherein the data label synchronization device is configured to establish links with the plurality of single-channel EEG acquisition devices based on Bluetooth through a connection parameter; wherein a connection period of each target link in the links is a baseline connection interval, and an interval between start moments of any two adjacent target links is a connection time threshold; wherein the connection parameter comprises a number of slave devices, the baseline connection interval and the connection time threshold; and
receive a start acquisition command sent by the data label synchronization device; wherein the start acquisition command is sent to each target single-channel EEG acquisition device by the data label synchronization device every time the data label synchronization device traverses a target single-channel EEG acquisition device in the plurality of single-channel EEG acquisition devices during traversing the plurality of single-channel EEG acquisition devices individually, after the data label synchronization device selects a baseline single-channel EEG acquisition device from the plurality of single-channel EEG acquisition devices based on an interrupt time interval corresponding to a monitored radio frequency mapping interrupt event;
wherein each target single-channel EEG acquisition device in the plurality of single-channel EEG acquisition devices performs acquisition at a radio frequency mapping interrupt of the target single-channel EEG acquisition device based on a target delay time period corresponding to the target single-channel EEG acquisition device, wherein the target delay time period is comprised in the received start acquisition command, to start a data acquisition at a target acquisition moment of a current first baseline connection interval of the baseline single-channel EEG acquisition device to obtain a plurality of target acquisition data of the target single-channel EEG acquisition device with an acquisition duration being the baseline connection interval individually, and each target single-channel EEG acquisition device performs data transmission at a radio frequency mapping interrupt of the target single-channel EEG acquisition device based on the corresponding target delay time period, to send obtained target acquisition data at a start moment of a next baseline connection interval of the target single-channel EEG acquisition device when obtaining each target acquisition data, wherein the target acquisition moment is a moment preceding a start moment of a next baseline connection interval of the baseline single-channel EEG acquisition device with a time interval of a preset acquisition time threshold, and the target acquisition data comprises a same number of data points.

12. The system according to claim 11, wherein each single-channel EEG acquisition device is further configured to perform one of:
after powering on and entering the broadcast mode in response to the setup operation of the user, and before receiving the start acquisition command sent by the data label synchronization device, in a case that the single-channel EEG acquisition device receives a first alignment reference time point sent by the data label synchronization device, determining the first alignment reference time point as a current time point of a second alignment timer, to realize time synchronization between the second alignment timer and the first alignment timer, wherein the first alignment reference time point is obtained by summing a first current time point with the baseline connection interval, and the first current time point is read after selecting the baseline single-channel EEG acquisition device from the single-channel EEG acquisition devices based on the interrupt time interval corresponding to the monitored radio frequency mapping interrupt event, and before traversing the single-channel EEG acquisition devices individually and sending the start acquisition command to the target single-channel EEG acquisition device for each target single-channel EEG acquisition device among the single-channel EEG acquisition devices traversed among the single-channel EEG acquisition devices; or
after determining the first alignment reference time point as the current time point of the second alignment timer, in a case that the single-channel EEG acquisition device receives a second alignment reference time point sent by the data label synchronization device and determines that a difference between the second alignment reference time point and the current time point of the second alignment timer is not greater than the baseline connection interval, replacing the current time point of the second alignment timer with the second alignment reference time point, wherein the second alignment reference time point is sent to the baseline single-channel EEG acquisition device by the data label synchronization device, and obtained by summing a second current time point of the first alignment timer with the baseline connection interval, wherein the second current time point is read by the data label synchronization device in a case that an interval between the current time point and a time point of sending the first alignment reference time point to the baseline single-channel EEG acquisition device is monitored to be increased by a preset calibration interval threshold.

13. (canceled)

14. The system according to claim 1, wherein each single-channel EEG acquisition device is preset with a corresponding fixing method; wherein the fixing method comprises a fixing method combining a hairpin wearing structure and an auxiliary electrode; wherein the hairpin wearing structure comprises a hardware fixing structure and a hairpin structure; wherein the fixing method is configured to fix hairs of a participant using a groove of the hairpin structure, the hardware fixing structure and the auxiliary electrode, and fix the single-channel EEG acquisition device on scalp of the participant to collect a EEG signal of the participant.

15. The system according to claim 14, wherein the hardware fixing structure comprises a housing, an acquisition electrode fixing structure, an internal circuit structure and a battery fixing structure; wherein the acquisition electrode fixing structure of the housing is a groove structure, the groove structure is configured to fix a replaceable electrode, and a groove of the groove structure comprises conductive tape or copper foil connected with a signal line of an acquisition circuit board; wherein the internal circuit structure of the housing is a three-layer structure, card structures are provided in the three-layer structure configured to place a power supply board, a battery module, and the acquisition circuit board respectively, and the card structures are provided with rectangular empty grooves configured to place a connection line between the acquisition circuit board and the power supply board; wherein the battery fixing structure of the housing is configured to place two electrodes of the power supply board; wherein a rectangular hole is provided on one side of the housing, and the rectangular hole is configured to connect the hairpin structure; wherein the hardware fixing structure is provided with a partition and a support structure on a third layer, for fixing the power supply board.

16. The system according to claim 15, wherein a fixing connection structure is provided on one side of the housing, wherein the hairpin structure is a dual-side flip structure, and comprises a hair fixing structure and a flip baseline structure, wherein the fixing connection structure is configured to connect the hair fixing structure; wherein the flip baseline structure comprises a semi-circular groove or a rotation pivot structure; wherein the hair fixing structure is provided with the groove, wherein a bottom part of the groove is a buckle, configured to lock the housing, wherein an upper part of the buckle is provided with a semi-cylindrical structure, configured to connect the housing, the hair and the hair fixing structure.

17. The system according to claim 16, wherein the replaceable electrode comprises an electrode body and an electrode substrate, wherein the electrode body comprises one of a disk electrode, an elastic copper ball electrode, or a sintered silver-silver chloride electrode; wherein the electrode substrate comprises a square electrode substrate, and the square electrode substrate is a printed circuit board (PCB) board; or
wherein the replaceable electrode is configured to fix the electrode substrate to the groove structure of the housing, to connect the PCB board to the copper foil connecting with the signal line of the acquisition circuit board in the groove structure.

18. (canceled)

19. A method for data processing, applied to a multi-channel miniature electroencephalogram (EEG) acquisition system, the system comprising:
a plurality of single-channel EEG acquisition devices, a data label synchronization device, and a terminal device, wherein the terminal device comprises a waveform recording module and a stimulation module;
wherein:
the stimulation module is configured to transmit stimulation label data to the data label synchronization device through a universal serial bus (USB) interface in a case that a stimulation signal is generated;
the data label synchronization device is configured to issue clock synchronization information and a control command, and receive the stimulation label data;
the plurality of single-channel EEG acquisition devices are configured to receive the clock synchronization information, collect EEG data based on the control command, and transmit the EEG data to the data label synchronization device through Bluetooth low energy (BLE);
the data label synchronization device is further configured to synchronize the EEG data and the stimulation label data, and transmit synchronized data and the stimulation label data to the waveform recording module; and
the waveform recording module is configured to display a waveform of the EEG data corresponding to the stimulation label data and perform data store;
wherein the method comprises:
obtaining stimulation label data in a case that a stimulation signal is generated;
obtaining clock synchronization information, performing clock synchronization, and obtaining a signal acquisition command;
collecting electroencephalogram (EEG) data based on the signal acquisition command after the clock synchronization, and synchronizing the EEG data and the stimulation label data; and
obtaining a waveform of the EEG data corresponding to the stimulation label data based on the synchronized data and storing the data.

20. The method according to claim 19, wherein the method is applied to the data label synchronization device, and the method further comprises:
establishing links with the plurality of single-channel EEG acquisition devices based on Bluetooth through a connection parameter; wherein a connection period of each target link in the links is a baseline connection interval, and an interval between start moments of any two adjacent target links is a connection time threshold; wherein the connection parameter comprises a number of slave devices, and the baseline connection interval and the connection time threshold;
selecting a baseline single-channel EEG acquisition device from the plurality of single-channel EEG acquisition devices based on an interrupt time interval corresponding to a monitored radio frequency mapping interrupt event; wherein the interrupt time interval represents a time interval between a current radio frequency mapping interrupt event and a previous radio frequency mapping interrupt event; wherein the baseline single-channel EEG acquisition device is a single-channel EEG acquisition device that first establishes a target link with the data label synchronization device during the baseline connection interval; and
traversing the plurality of single-channel EEG acquisition devices individually, and when traversing each target single-channel EEG acquisition device among the plurality of single-channel EEG acquisition devices, sending a start acquisition command to the target single-channel EEG acquisition device, wherein each target single-channel EEG acquisition device performs acquisition at a radio frequency mapping interrupt of the target single-channel EEG acquisition device based on a target delay time period corresponding to the target single-channel EEG acquisition device, wherein the target delay time period is comprised in the received start acquisition command, to start a data acquisition at a target acquisition moment of a current first baseline connection interval of the baseline single-channel EEG acquisition device to obtain a plurality of target acquisition data of the target single-channel EEG acquisition device with an acquisition duration being the baseline connection interval individually, and each target single-channel EEG acquisition device performs data transmission at a radio frequency mapping interrupt of the target single-channel EEG acquisition device based on the corresponding target delay time period, to send obtained target acquisition data at a start moment of a next baseline connection interval of the target single-channel EEG acquisition device when obtaining each target acquisition data, wherein the target acquisition moment is a moment preceding a start moment of a next baseline connection interval of the baseline single-channel EEG acquisition device with a time interval of a preset acquisition time threshold, and the target acquisition data comprises a same number of data points.

21. The method according to claim 19, wherein the method is applied to the plurality of single-channel EEG acquisition devices, and the method further comprises:
powering on and entering a broadcast mode in response to a setup operation of a user, wherein the data label synchronization device to establishes links with the plurality of single-channel EEG acquisition devices based on Bluetooth through a connection parameter; wherein a connection period of each target link in the links is a baseline connection interval, and an interval between start moments of any two adjacent target links is a connection time threshold; wherein the connection parameter comprises a number of slave devices, the baseline connection interval and the connection time threshold;
receiving a start acquisition command sent by the data label synchronization device; wherein the start acquisition command is sent to each target single-channel EEG acquisition device by the data label synchronization device every time the data label synchronization device traverses a target single-channel EEG acquisition device in the plurality of single-channel EEG acquisition devices during traversing the plurality of single-channel EEG acquisition devices individually after the data label synchronization device selects a baseline single-channel EEG acquisition device from the plurality of single-channel EEG acquisition devices based on an interrupt time interval corresponding to a monitored radio frequency mapping interrupt event; and
performing, by each target single-channel EEG acquisition device in the plurality of single-channel EEG acquisition devices, acquisition at a radio frequency mapping interrupt of the target single-channel EEG acquisition device based on a target delay time period corresponding to the target single-channel EEG acquisition device, wherein the target delay time period is comprised in the received start acquisition command, to start a data acquisition at a target acquisition moment of a current first baseline connection interval of the baseline single-channel EEG acquisition device to obtain a plurality of target acquisition data of the target single-channel EEG acquisition device with an acquisition duration being the baseline connection interval individually, and each target single-channel EEG acquisition device performs data transmission at a radio frequency mapping interrupt of the target single-channel EEG acquisition device based on the corresponding target delay time period, to send obtained target acquisition data at a start moment of a next baseline connection interval of the target single-channel EEG acquisition device when obtaining each target acquisition data is obtained, wherein the target acquisition moment is a moment preceding a start moment of a next baseline connection interval of the baseline single-channel EEG acquisition device with a time interval of a preset acquisition time threshold, and the target acquisition data comprises a same number of data points.

22. The method according to claim 19, further comprising:
obtaining a fixing method for each single-channel EEG acquisition device; wherein the fixing method comprises a fixing method combining a hairpin wearing structure and an auxiliary electrode; wherein the hairpin wearing structure comprises a hardware fixing structure and a hairpin structure; and
fixing hairs of a participant according to the fixing method using a groove of the hairpin structure, the hardware fixing structure, and the auxiliary electrode, and collecting an EEG signal of the participant by fixing each single-channel EEG acquisition device on scalp of the participant.

23. An electronic device, comprising: a processor, and a memory storing an embedded program executable by the processor, wherein the processor is configured to perform a method for data processing applied to a multi-channel miniature electroencephalogram (EEG) acquisition system, the system comprising:
a plurality of single-channel EEG acquisition devices, a data label synchronization device, and a terminal device, wherein the terminal device comprises a waveform recording module and a stimulation module;
wherein:
the stimulation module is configured to transmit stimulation label data to the data label synchronization device through a universal serial bus (USB) interface in a case that a stimulation signal is generated;
the data label synchronization device is configured to issue clock synchronization information and a control command, and receive the stimulation label data;
the plurality of single-channel EEG acquisition devices are configured to receive the clock synchronization information, collect EEG data based on the control command, and transmit the EEG data to the data label synchronization device through Bluetooth low energy (BLE);
the data label synchronization device is further configured to synchronize the EEG data and the stimulation label data, and transmit synchronized data and the stimulation label data to the waveform recording module; and
the waveform recording module is configured to display a waveform of the EEG data corresponding to the stimulation label data and perform data store;
wherein the method comprises:
obtaining stimulation label data in a case that a stimulation signal is generated;
obtaining clock synchronization information, performing clock synchronization, and obtaining a signal acquisition command;
collecting electroencephalogram (EEG) data based on the signal acquisition command after the clock synchronization, and synchronizing the EEG data and the stimulation label data; and
obtaining a waveform of the EEG data corresponding to the stimulation label data based on the synchronized data and storing the data.
